(19) United States
(12) Patent Application Publication
(10) Pub. No.: US 2025/0041207 A1
(43) Pub. Date: Feb. 6, 2025

(54) TREATMENTS FOR UTERINE FIBROIDS

(71) Applicant: RESURGE THERAPEUTICS, INC., San Jose, CA (US)

(72) Inventors: Olof Mikael TROLLSAS, San Jose, CA (US); John J. STANKUS, Santa Cruz, CA (US); Shahram Shawn GHOLAMI, Monte Sereno, CA (US)

(21) Appl. No.: 18/362,383

(22) Filed: Jul. 31, 2023

(51) Int. Cl. A61K 9/00; A61K 31/337; A61K 31/436; A61K 47/34

(52) U.S. Cl. CPC A61K 9/0024; A61K 31/337; A61K 31/436; A61K 47/34

(57) ABSTRACT

Minimally invasive treatment methods for uterine fibroids. A system includes a sustained release formulation comprising a cytostatic or cytotoxic drug, and an applicator or delivery system for local delivery of a composition comprising or consisting essentially of the sustained release formulation to the uterus.

### FIELD

The present disclosure relates to compositions, systems, instruments, methods, and processes, for minimally invasive, local treatments for women's health and, more particularly, uterine fibroids.

### BACKGROUND

The most common types of uterine treatment are treatments for non-cancerous conditions (i.e., non-malignant tumors) and enlarged uterus, more commonly known as uterine fibroids. Uterine fibroids are knots of benign smooth muscle in the uterine wall. They are symptomatic in at least 25% of all women, likely 40-80% of women have them and they are the leading cause for hysterectomy, accounting for more hysterectomies than all gynecologic cancers combined. Their most common symptoms are heavy or long menstrual bleeding (menorrhagia) and pelvic pressure or discomfort. Typical menstrual periods last 4-5 days while menorrhagia lasts for 7 days or more. Short excessively heavy periods can also occur with menorrhagia. These symptoms lead to significant negative quality of life symptoms such as excessive pad and tampon changing and usage, wearing of adult diapers, flooding or loss of menstrual control leading to staining of clothes, loss of sleep having to get up at night to change pads and/or tampons. Fibroids can also lead to anemia or low red blood cell count which makes patients feel tired or weak.

There is an unmet clinical need to treat uterine fibroids with improved and sustained efficacy, administered via a less invasive procedure and with less associated side effects.

### SUMMARY

The present disclosure provides compositions, systems, methods of administration, and processes, for local delivery of a sustained release formulation (SRF) for efficacious treatment of a target uterine tissue, and/or providing relief from symptoms originating from or associated with uterine fibroids while mitigating, if not avoiding, damage to nearby uterine structures or the ovaries. The treatment may be used by itself or in combination with other known treatments.

### BRIEF DESCRIPTION OF THE DRAWINGS

FIG. 1 is a PRIOR ART perspective view of a needle syringe.

FIGS. 2A, 2B are schematic side-view illustrations of needle syringes for delivering a composition comprising a sustained release formulation (SRF).

FIGS. 3A, 3B, and 3C provide examples of H-Injectate Ratings (HIRs) for treatment of Uterine Fibroids.

FIG. 4 shows Drug Release Curves for SRFs A, B and C.

### DETAILED DESCRIPTION

The present disclosure provides compositions, systems, methods of administration, and processes, for local delivery of a sustained release formulation (SRF) for efficacious treatment of a target uterine tissue, and/or providing relief from symptoms originating from or associated with uterine fibroids while mitigating, if not avoiding, damage to nearby uterine structures or the ovaries. The treatment may be used by itself or in combination with other known treatments.

The tissue types are very different between treatments for cancer or pre-cancerous tumors versus fibroids, which calls for different formulations of a similar drug that may be effective in treating either condition. Most notably, the volume of drug (on a per-volume of uterus basis) is very different when treating a tumor or cancer than when treating fibroids. Finally, when treating for cancer or tumor one wants to have the full drug release from a carrier quickly, i.e., within the first 24 hours of injecting the composition into the patient. A slow release, in contrast, is intended to mean a full drug release at a minimum of 1-3 months' time from when the composition was injected into the patient.

Procedures for treating uterine fibroids have demonstrated varying levels of efficacy and are often accompanied by undesirable or adverse effects. Surgical treatments for fibroids include myomectomy, hysterectomy, uterine fibroid embolization, radiofrequency ablation and MRI guided ultrasound. Myomectomy is a surgery where fibroids are removed without damaging the uterus which is attractive for patients that desire future pregnancy. Myomectomy may include hysteroscopy where a scope is inserted through the vagina to the uterus, laparoscopy where a scope is inserted in small incisions in the abdomen and potentially assisted with a robot, and laparotomy where a larger cut is made in the abdomen. Hysteroscopy is typically more appropriate with smaller intracavity and submucosal fibroids and is associated with a high 15-50% risk of recurrence. The cumulative effect of possible multiple future surgeries must be considered by the patient. Abdominal myomectomy (laparoscopic, robotic, or laparotomic) can reduce intramural, subserosal and very large submucosal fibroids typically not amenable via hysteroscopy. A disadvantage of myomectomy is that patients in the future may need to deliver a baby via Caesarean section.

For patients who don't desire future pregnancy surgical treatments may include hysterectomy where the entire uterus is removed. This surgery is the only current approach to cure fibroids and prevent their recurrence though may have long term health effects. There are also vaginal, laparoscopic and robotic approaches to hysterectomy. If the uterus alone is removed and ovaries are left in place, the patient will not go into menopause after a hysterectomy. Typically, the least invasive approach is utilized for hysterectomy when possible. Fibroids account for approximately one third of all hysterectomies. Long term risks of hysterectomy (especially in conjunction with oophorectomy or ovary removal) include increased risk of cardiovascular disease, fractures, pelvic floor dysfunction, and neurological issues.

Less invasive targeted drug delivery approaches to the uterus zone have been attempted by the transvaginal or transcervical routes. Given the invasiveness of surgery, many women desire a treatment that is less invasive while still preserving their uterus for possibility of future pregnancy. Other less invasive approaches include uterine fibroid embolization (UFE), radiofrequency (RF) ablation, and MRI guided ultrasound. UFE is when an interventional radiologist working together with a gynecologist uses a catheter in the uterine artery or radial artery and embolic particles are delivered to block the flow of blood from the uterine artery to the fibroids. Loss of blood flow shrinks the fibroids, improving symptoms, while at the same time sparing non-involved uterus. UFE is recommended for patients who are not good surgical candidates or want to avoid surgery. It can be performed under intravenous and local anesthesia. It is applicable for multiple fibroids and very large fibroids. Disadvantages include a painful though relatively short 5-7 days recovery, 17% chance of reintervention, chance of damage to uterus and other unwanted embolization, post embolization syndrome and it may negatively impact uterine, ovarian function and fertility so that typically it is not an option for patients desiring future pregnancy.

Radiofrequency (RF) ablation uses RF energy to thermally ablate and necrose the fibroids and can be delivered by laparoscopic, transvaginal or transcervical approaches as an alternative to surgery. For example, the Acessa procedure is performed via laparoscopic small abdominal incisions. The SONATA system by Gynesonics is an example of a RF product for fibroids that is incisionless, delivered via concurrent ultrasound imaging and preserves the uterus via delivery from a transvaginal and transcervical approach. The transcervical device contains both the ultrasound probe and RF electrodes in a single delivery device to optimize fibroid imaging in the same plane. The RF ablation volume can be tailored to the fibroid size. It has shown demonstrated improvements in bleeding and patient quality of life out to 3 years in controlled clinical trials. However, it still requires use of general anesthesia and recovery takes 10-14 days and typically can only treat fibroids less than 5 cm in size. Similar to UFE it may negatively impact uterine, ovarian function and fertility so that typically it is not an option for patients desiring future pregnancy. Another disadvantage of focused energy based approaches used to desiccate or ablate fibroids is that they treat them one at a time and target the center of fibroids where fibroids typically growth from their periphery.

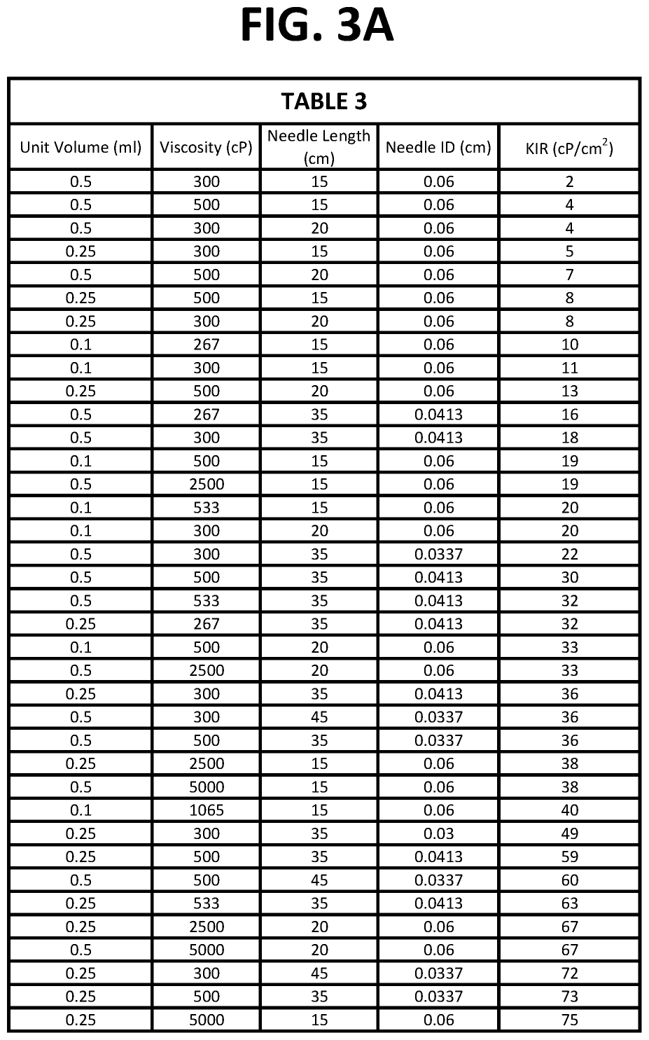

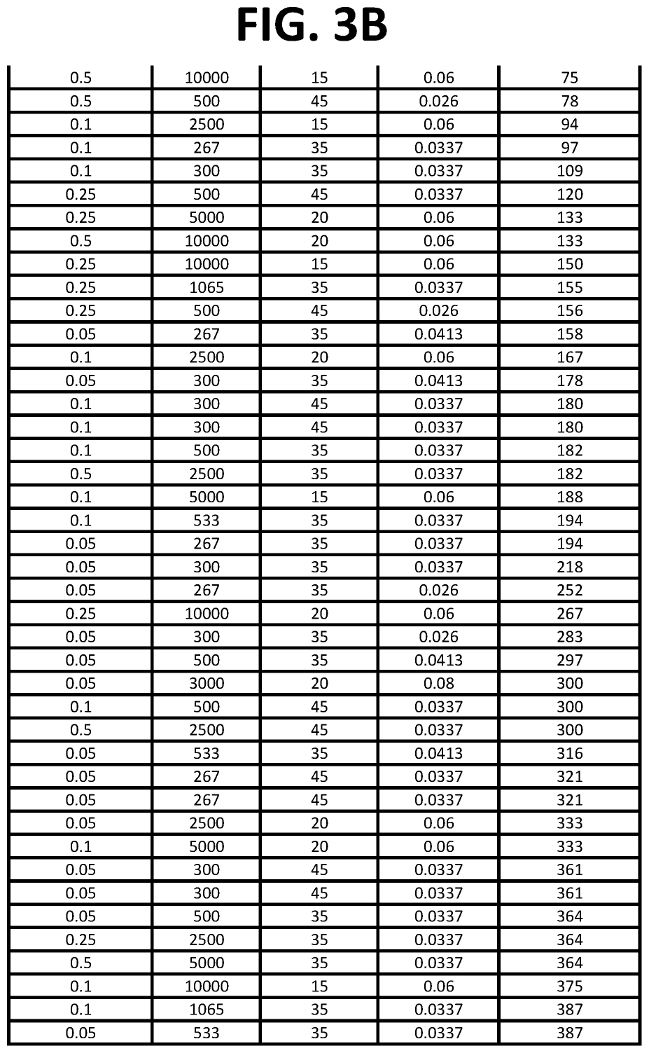

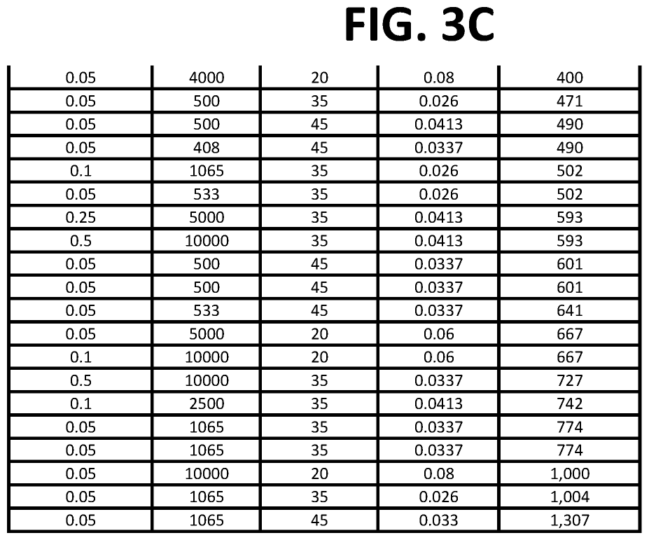

MRI guided high energy focused ultrasound is delivered with the patient inside the MRI machine where then ultrasound sends targeted soundwaves to ablate and necrose the fibroids. Disadvantages of HIFU include long treatment time, poor patient tolerance and a third of women will need another future procedure due to recurrence. Similar to UFE it may negatively impact uterine, ovarian function and fertility so that typically it is not an option for patients desiring future pregnancy. To improve on HIFU, local transvaginal injection of ethanol in conjunction with oxytocin intravenous drip have been investigated (Zhang et al.). Transvaginal needle injection in conjunction with transrectal ultrasound imaging is shown in FIG. 3. Note for needle based procedures in the uterus such as biopsy needle sizes typically are 14-20 gauge in diameter and 10-40 cm in length approximately (Ferroro et al. 2019).

While the foregoing methods may show efficacy in reducing fibroids, they either require a more invasive procedure (versus localized treatment using the delivery device as disclosed herein), more frequent treatment due to diffusion or more generalized treatment of fibroids raising the possibility of adverse effects because a comparatively high dosage of the drug (i.e., at least 2 to 3 times higher % vol. compared to the formulations used for an SRF) is needed to treat the area while accounting for leakage or diffusion of the drug to other areas. Adverse effects may include diminished urinary or sexual function. It is desired to have an effective treatment targeting only the target fibroid tissue and nowhere else (e.g., avoiding the bladder, healthy uterus, ovaries and nerves surrounding the uterus) and to perform the procedure in a less invasive manner for patient acceptance.

### Definitions

Unless otherwise specified, all terms of art, notations and other scientific terminology used herein are intended to have the meanings commonly understood by those of skill in the art to which this invention pertains. For purposes of this disclosure, the following terms and definitions apply:

The terms “about” or “approximately” is defined herein as 30%, 20%, 15%, 10%, 5%, 4%, 3%, 2%, 1.5%, 1%, between 1-2%, 1-3%, 1-5%, or 0.5%-5% less or more than, less than, or more than a stated value, a range or each endpoint of a stated range, or a one-sigma, two-sigma, three-sigma variation from a stated mean or expected value (Gaussian distribution). For example, d1 is about d2 means d1 is 30%, 20%, 15%, 10%, 5%, 4%, 3%, 2%, 1.5%, 1%, 0% or between 1-2%, 1-3%, 1-5%, or 0.5%-5% different from d2. If d1 is a mean value, then d2 is about d1 means d2 is within a one-sigma, two-sigma, or three-sigma variance from d1. It is understood that any numerical value, range, or either range endpoint (including, e.g., “approximately none”, “about none”, “about all”, etc.) preceded by the word “about,” “substantially” or “approximately” in this disclosure also describes or discloses the same numerical value, range, or either range endpoint not preceded by the word “about,” “substantially” or “approximately.”

The terms “drug” or “agent” as used herein is defined as a therapeutic substance, other than food, used in the prevention, diagnosis, alleviation, treatment, or cure of disease. Unless stated otherwise, “drug” and “agent” shall have the same meaning.

The term “cytostatic” as used herein refers to a drug that is non-toxic to cells but does mitigate cell proliferation and permit cell migration. Cytostatic drugs may include without limitation rapamycin, sirolimus, everolimus, zotarolimus, myolimus, temsirolimus, tacrolimus, macrolide antibiotics, ridaforolimus, biolimus, novolimus, deforolimus, structural derivatives and functional analogues of rapamycin and any macrolide immunosuppressive drug. mTOR/PI3K dual inhibitors may also be utilized including dactolisib, BGT226, SF1126, PKI-587, and NVPBE235, mTORC1/mTORC2 dual inhibitors may also be utilized including sapanisertib, AZD8055, AZD2014 as derived from morpholino pyrazolopyrimidine.

The term “cytotoxic” as used herein refers to a drug that inhibits cell growth and proliferation such as chemotherapeutics. These drugs may include but are not limited to paclitaxel, taxanes, protaxel, vincristine, etoposide, nocodazole, indirubin, anthracycline derivatives, daunorubicin, daunomycin, tauromustine, bofumustane, carboplatin, carmustine, cisplatin, docetaxel, gemcitabine, mitomycin, procarbazine, and plicamycin. These drugs may also be apoptotic such as TGF, topoisomerase inhibitors, including, 10-hydroxycamptothecin, irinotecan, and doxorubicin.

The term “composition” as used herein means a product of mixing or combining various elements or ingredients. Whenever the word “composition” is used, it will be understood that the composition comprises an SRF. The term “polymer composition” however is not a composition that comprises the SRF.

The term “absolute viscosity” as used herein means the viscosity measured relative to the viscosity of a known substance. Unless stated otherwise “absolute viscosity” (as compared to inherent viscosity) is represented by the symbol p. Absolute viscosities may be measured using a calibrated digital rotational viscometer spindle (no. 1 size) with 10 mL solutions in 40 ml polypropylene vials with an approximately 50% submerged spindle solution contact, with measurements made at room temperature conditions.

The term “target tissue” as used herein is defined as uterine tissue to include the endometrium, submucosal, myometrium, intramural, serosa and subserosal locations of the uterus. In certain embodiments, the “target tissue” is a uterine fibroid.

The present disclosure includes polymer nomenclature whereby the polymer is defined, followed by the monomer ratio and a specification of any end groups. The following are examples of the polymer naming nomenclature appearing in the listing of additional disclosed embodiments following the detailed description. Other examples not explicitly spelled out here use the same rationale: PLGA8515A (0.3 dl/g) means poly(lactide-co-glycolide) with a monomer ratio of 85/15, end capped with an acid group (A), and an inherent viscosity of 0.3 dl/g; and PLGA6535E (0.5 dl/g) means poly(lactide-co-glycolide) with a monomer ratio of 65/35, end capped with an ester groups (E), and an inherent viscosity of 0.5 dl/g; and PLGA5050A (0.2 dl/g) means poly(lactide-co-glycolide) with a monomer ratio of 50/50, end capped with an acid group (E), and an inherent viscosity of 0.2 dl/g; and Poly(lactide-co-glycolide) is typically poly(D,L-lactide-co-glycolide) but could also be e.g. any or a mixture of poly(D,L-lactide-co-glycolide), poly(D-lactide-co-glycolide), and poly(L-lactide-co-glycolide).

The term “sustained release formulation (SRF)” as used herein refers to a substance for treating uterine fibroids, the substance including a drug (or drugs), solvent for the drug and carrier for the drug(s) or drug carrier comprising a polymer composition administered to the target tissue in liquid, gel or solid form using a delivery vehicle, such as a needle syringe, whereupon local delivery of the composition (comprising the SRF) to the target tissue the SRF is effective in producing a sustained release of the drug(s) to the targeted tissue of the uterus, thereby producing an efficacious result over a period of time, e.g., from 14 days, 30 days, 1 to 3 months, up to 6 months, up to 12 months, or up to 2 years following treatment and with minimal uncontrolled drug diffusion. Other substances may also be present in the composition (e.g., an ultrasound/echoing enhancing medium or other imaging enhancing depending on the imaging modality used). A programmed, sustained release of, e.g., from 14 days or 1 to 12 months for substantially all the drug to dissipate from the carrier is achieved by selection of drug carrier (polymer composition) and/or modifying the morphology and mechanical properties, the polymer/drug ratio, and controlling the physical shape/dimensions (volume) of the SRF and/or composition that is delivered to the target tissue. Other factors influencing a release rate are described in greater detail, below.

Given the existing state of the art for treating uterine fibroids, the inventors sought to develop a formulation that could be delivered by way of local uterine needle injection and that could overcome the foregoing drawbacks with existing procedures for treating fibroids. The formulation developed is the SRF, which is described in greater detail below, followed by several embodiments of the SRF. Embodiments of a device and method for delivering the SRF using the device, which involves use of a needle syringe, are also described.

Note smaller fibroid or fibroid seedlings that have higher growth rates have been found to have higher amounts of proliferating smooth muscle cells. Therefore, the inventors recognized that anti-proliferative drugs that target active proliferating smooth muscle cells could be a novel therapy for treatment of seedling fibroids and smaller fibroids prior to progression to larger ones. The inventors also recognized that when fibroids grow large enough the cores can necrose with proliferation occurring mostly on the peripherally, which requires an anti-proliferative therapy that can sufficiently circumferentially cover and penetrate to treat the fibroid exterior.

The question asked was whether the SRF could be delivered to the uterus on a consistent, repeatable basis to ensure a total volume by uterus size is substantially met, but not exceeded, by an administering health professional, to achieve the desired outcome (reduction in uterus volume, over a period of time by a programmed, sustained release of a drug) and without adverse indications (e.g., infection, post-treatment pain or discomfort, drug diffusion into nearby areas such as the ovaries, rectum or bladder). The inventors found, after extensive bench testing and evaluation of several trials of both delivery device and candidate SRF formulations, a needle syringe suited to deliver the SRF in the manner sought by the inventors.

Disclosed herein is a method for delivery of a SRF in an efficacious manner and with less side effects to a patient. The method uses an apparatus comprising a needle syringe containing the SRF. The method includes dispensing one or more unit volumes of composition from the needle syringe at a respective one or more target tissue locations of the uterus. The step of injecting the composition can be performed repeatedly using the same needle syringe. The apparatus is characterized by its H-injectate Rating (HIR) defined by the following relationship:

$$HIR = \frac{μ {L}^{2}}{vD} ( 1 {0}^{- 6} )$$

Where μ is the absolute viscosity (cP) of the composition, L is the needle length (cm) measured from the needle tip to the end of the needle hub, D is the needle lumen diameter (cm), and v is a unit volume (cm³ or cc) of the composition contained in the syringe barrel. Each unit volume represents a maximum volume of the composition dispensed at a single location of the target tissue. In some embodiments the syringe holds a plurality of unit volumes, which enables the dispensing of a unit volume at each of a corresponding plurality of discrete locations of the target tissue. Each fibroid based on its fibroid volume would receive 1 or more unit volumes. Unit volumes are necessary for the backflow and tissue loss issues associated with larger single volumes of injections. In certain embodiments, the total volume delivered per uterus would be a summation of one or more fibroids treated of similar or different sizes.

A patient can have one or more fibroid volumes each being either similar or different such that a total SRF volume would be based on a summation of each fibroid volume.

There is also disclosed an apparatus for uterine treatment. The apparatus comprises a needle syringe and the composition contained within the syringe. The apparatus is adapted to deliver the composition in one or more unit volume increments to a target tissue, as defined by its HIR value.

HIR values for the method, apparatus, medical device, and system according to the disclosure may range from between 2 and 1300, 10 and 300, or 40 and 400. The units of HIR are centipoise per unit area.

Uterine treatment according to the disclosure may be described as a fibroid treatment having three characteristics: minimal uncontrolled drug diffusion, sustained release and efficacy, and a unit volume as defined herein. The HIR identifies the needle syringe capable of delivering these one or more unit volumes of a composition, in a safe, repeatable pattern by a health professional and without imposing significant compromises on the composition's ability to achieve minimal uncontrolled drug diffusion, sustained release and efficacy.

Accordingly, in one aspect, a treatment of uterine fibroid tissue, as provided herein, includes the delivery of a drug or multiple drugs to the tissue in a sustained release manner using a needle syringe containing the SRF. The treatment may be used with, or in addition to treatments involving removal/ablation of tissue, and/or delivery of energy to the tissue and additionally the administering of various agents. Methods according to the disclosure may additionally, or alternatively, be administered after a treatment of fibroids according to other methods.

Access to uterine tissue may be achieved in a transvaginal or laparoscopic manner. It may be beneficial and less invasive to access the tissue by either transvaginal or mini laparoscopic approaches. The advantages with these approaches include one or more (1) oral and/or local anesthesia application instead of general anesthesia, (2) less trauma and less resulting side effects, (3) faster recovery time for the patient, (4) familiar treatment for the gynecologist physician similar to uterus biopsy. For access by transvaginal approach, guidance may be provided by optical, ultrasound, x-ray, computed tomography, magnetic resonance imaging or other imaging modality. Ultrasound imaging may be beneficial given that ultrasound is utilized for uterus biopsy. The transvaginal approach closely mirrors the present ultrasound and biopsy techniques familiar to gynecologists. Transvaginal effects avoid the need to create a surgical incision in the abdomen and allows for direct visualization and access to submucosal fibroids in the uterus.

The drug portion of the composition may be an anti-inflammatory, anti-proliferative, cytoreductive, cytostatic, and/or cytotoxic drug that would affect the fibroid size and proliferation. The apparatus enables delivery of one or more drugs into the target tissue. Once delivered to the target tissue, the drug may then release from the SRF in a slow, sustained release fashion, optionally delivered as an initial burst of the drug, followed by a slow, sustained release of the drug to the target release. The amount or lack of burst and/or the “slow, sustained release” release period may depend on the drug delivered to the uterus, the SRF properties, and the HIR value, as will be appreciated in view of this disclosure. In some embodiments a slow, sustained release may occur over, e.g., a 24-hour period, 3-7 days, 1-4 weeks, 1 to 12 months, 3 months, or 6 months.

In another aspect there is a system for treating fibroids including a needle syringe, the sustained release formulation (SRF), and imaging device for locating a target tissue of the uterus. The imaging device, e.g., ultrasound, may be used both as a needle guide to the target tissue and for sizing the uterus/fibroids. Once the fibroids number, sizes and locations are determined, the number, n, and size of unit volumes, v, for injection may be determined. A needle syringe according to the disclosure may hold one or a plurality of unit volumes dispensable from the needle to treat the fibroids n a controlled manner, as determined by the HIR value for the syringe containing the composition. The HIR value is between 2 and 1307, 10 and 300, or 40 and 400.

In another aspect there is a method for making a medical device for treating the uterus, including the steps of combining at least one drug with at least one polymer carrier and a solvent to form a composition comprising an SRF, wherein the composition is contained within a needle syringe, and wherein the needle syringe containing the composition has a HIR value of between 2 and 1307, 10 and 300, or 40 and 400.

Treatment of uterine fibroids by a needle injection, in a small quantity (unit volume) at a plurality of locations in the target tissue, offers several benefits over other methods. Among the benefits are less invasive procedures leading to greater patient acceptance and less complications during patient treatment, less frequent procedures needed, and reduced incidence of drug affecting nearby tissue leading to such outcomes as adverse consequences for ovarian, urinary or sexual function.

The term “unit volume” as used herein refers to the maximum volume (v) of composition for an individual injection when treating uterine fibroids. In most cases there is a plurality of unit volumes of a composition per patient. For example, there can be n=2, 3, 4, 5, 6, or more, depending on each fibroid volume, which can be determined from ultrasound imaging prior to treatment. Herein, n is the number of unit volumes. The larger the size and number of the fibroid, generally speaking, the higher the number of unit volumes dispensed from a needle at different locations of the uterus. In some embodiments, the larger the size of the fibroid, the larger the unit volume. It was found in bench studies that a uterus treatment using a unit volume of 0.05 ml to 0.1 ml of composition can result in significant reduction in fibroid size over a 90 day period. Moreover, it is believed that a unit volume of 0.05 ml to 0.5 ml, 0.05 ml to 0.1 ml, 0.1 to 0.2, or 0.05 to 0.2 ml will also produce positive clinical outcomes with no adverse indications.

A uterine fibroid volume (FV) can range from 0.05 to 15 cc or more. Large fibroids can be 5-10 cm. For these sizes a larger volume of the SRF (e.g., 0.1, 0.5 or 0.2 ml) and/or more injections are needed. For medium fibroids, e.g., 5 cm, less volume can be needed (e.g., 0.07, 0.09, or 0.11 mL). To achieve approximately a >25% reduction in size, the number (n) of unit volumes (v) injected per fibroid may be selected based on uterus size. Therefore, to reduce a larger size uterus by the same amount, an increased number of unit volumes may be utilized. For example, the number (n) of unit volumes injected to achieve greater than 15%, or greater than 25% reduction in uterus size may be determined from TABLE 1.

TABLE 1

| Fibroid Volume | # Unit Volumes Injected Total |
|---|---|
| (cc) | (in each fibroid) |
| 0.05-1 | 1 |
| 1-5 | 1-2 |
| 5-10 | 2-3 |
| 10-15 | 3-4 |

The term “total volume” of composition (i.e., the sum of n unit volumes of composition injected into the uterus during a single treatment session, defined as within a 1-2 hours of, or within 24 hours of each other) is the total volume of composition administered to the patient and expected to produce a programmed, sustained release and efficacious outcome when treating uterine fibroids. In certain embodiments, the total volume is an effective amount of the sustained release formulation suitable for treating a subject in need thereof. This programmed, sustained release and efficacious outcome may be measured with menstrual blood loss volume, or the Uterine Fibroid Symptom-Quality of Life (UFS-QoL) include heavy bleeding, passing blood clots, duration of menstrual period, pelvic pressure, frequent urination, and fatigue. A total volume is expected to produce more than a 15% reduction in fibroid volume, or more than a 25% reduction in fibroid volume.

In some embodiments a patient to be treated has cancer in another part of the body (other than the uterus) but is being treated for fibroids according to the disclosure. In some embodiments the patient does not have cancer in another part of the body and is being treated for fibroids.

A SRF for treating fibroids, according to the disclosure, is preferably delivered transvaginally, transcervically, transuterine laparoscopically, and/or transrectally to the target tissue using a needle syringe having a relatively long and narrow needle, flexible needles included (for example, a 15 to 45 cm in length needle, and 0.06 cm to 0.12 cm needle lumen, alternatively a needle having a slenderness ratio, defined as needle length divided by lumen diameter, of between 125 and 750 or, more preferably, 400 and 750). Injections may be made via hysteroscope and robotic (in addition to laparoscopic) approaches. The fluid properties and injectate volume requirements of the composition using this type of needle were initially examined to determine whether there were any limitations, special considerations or modifications needed to the SRF (and/or delivery device) for delivery of this injectate to a uterus.

A uterine treatment according to the disclosure may be described as a uterus treatment having three characteristics:

1. MINIMAL UNCONTROLLED DRUG DIFFUSION: Obtaining sufficient solidification or gelation of the SRF in a relatively short period of time upon contact with the uterus, so that the SRF remains at the target tissue when the needle is removed and, hence, little or no drug diffusion takes place possibly affecting unintended, adjacent tissue such as the urethra or bladder. It is desirable to minimize an uncontrolled bolus of drug diffusion into surrounding tissue.

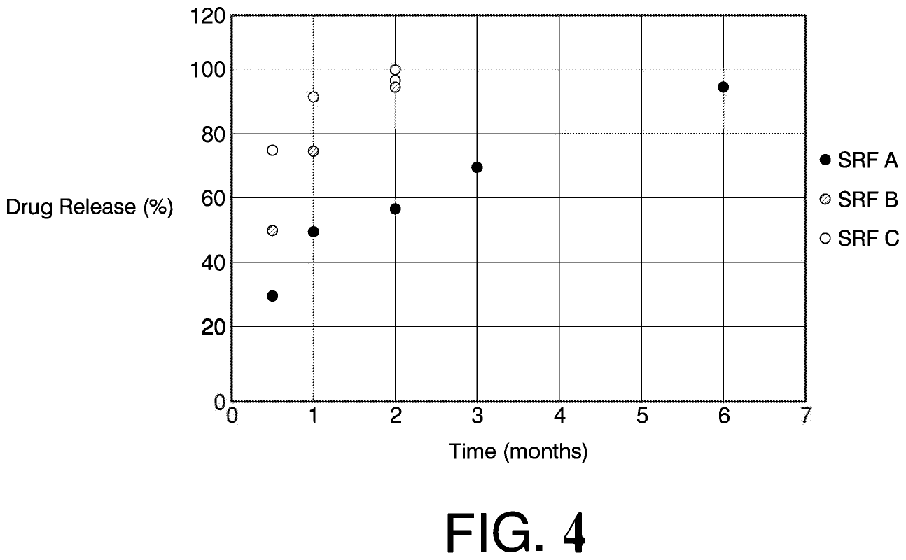

2. SUSTAINED RELEASE & EFFICACY: Enabling a programmed release of a drug over time from the SRF (see e.g., FIG. 4, TABLES 4A and 4B). The SRF is delivered in a sufficient total volume to enable a sustained release of a drug for prolonged treatment of the target tissue to achieve the efficacious result of reduced uterus size.

3. LOW UNIT VOLUME: Delivery of the composition total volume to the fibroid as one or more low unit volumes (i.e., “unit volume” as defined herein) using a needle syringe. This manner of delivery avoids causing additional acute pressure on the fibroid and urethra, which may cause additional discomfort for the patients immediately after or shortly after the procedure, or to avoid excessive swell due to uptake of fluids from the surrounding tissue. Additionally, a low unit volume of the composition avoids pressure at the injection site, which can mitigate against composition flow to surrounding tissues and organs and/or backflow of the composition through the delivery device or through the needle track once the needle is removed, and/or inhibiting the composition from being expelled from the target tissue due to pressure buildup. It can also be desirable to have a low unit volume injected at several discrete, nearby locations to achieve the targeted clinical outcome, as this can optimize diffusion of a hydrophobic drug relative to diffusion from a large volume injected in one location.

The factors influencing, and manner of arriving at the SRF formulation and composition enabling a uterus treatment with these characteristics will now be discussed. More specific examples of embodiments of an SRF are provided later.

Drug to Polymer Ratio. The drug to polymer ratio for the SRF formulation was investigated, as well as the drug to solvent ratio. Too low of a drug to polymer ratio is not attractive. If the drug dosage is too low, then it results in the local drug concentrations being too low. Too high of a drug to polymer ratio results in the drug releasing from the SRF too fast which limits the longer term local drug release and leads to surrounding tissue and/or systemic drug exposure.

Polymer to Solvent Ratio and Polymer Molecular Weight. The polymer to solvent ratio and polymer molecular weight associated with the SRF formulation were investigated. Too low of a polymer to solvent ratio is not attractive. If the polymer concentration and or molecular weight are too low, then it results in slow gelation and the drug releasing from the SRF too fast which limits the longer-term local drug release and leads to surrounding tissue and/or systemic drug exposure. As discussed in greater detail below, the desire to achieve a high gelation rate was balanced against the need for deliverability using a needle characterized by a high slenderness ratio. Too high of a polymer to solvent ratio and molecular weight results in a composition having a high viscosity, which may be delivered safely when using needles characterized by low slenderness ratios and in relatively large volumes. But the same polymer to solvent ratio and molecular weight can pose a significant challenge to deliver in unit volume amounts using needles characterized by a high slenderness ratio, in addition to the challenge of determining whether that selected polymer to solvent ratio and molecular weight produces the desired gelation rate without also producing too-slow of a drug release. Further, too high a viscosity presents a challenge for sufficient fibroid circumferential SRF coverage and penetration. It was these types of trade-offs or balances that led the inventors to identify an optimal combination of needle and composition, which is represented by the HIR.

It should be noted that the above-mentioned ratios and polymer molecular weights (examples provided below) are easily determined for the clinical use intended here; rather, it required lengthy investigation and discovery to arrive at the desired localized efficacy and effectiveness specifically tailored to satisfy all three characteristics of uterus treatment.

It is desired to have a programmed, sustained release of, e.g., from 14 days or 1 to 12 months for substantially all the drug to dissipate from the carrier. This requires the selection of the drug carrier (polymer composition) and/or modifying the morphology and mechanical properties, the polymer/drug ratio, controlling the physical shape/dimensions (volume) of the SRF and/or composition delivered to the target tissue. Other factors affecting the release rate include:
ability of polymer to swell (controlled by polymer structure e.g., monomers selection, monomer ratios, molecular weight, end groups, and porosity/morphology).
porosity/morphology (controlled by e.g., polymer structure and concentration, polymer/solvent ratio and miscibility, and polymer/drug ratio—less drug than polymer than drug is trapped).
how fast the materials gel and whether above or below wet glass transition temperature—transition from liquid to solid using water soluble or insoluble solvents (fast gelation leads to faster initial release/burst) (controlled by e.g., polymer structure and concentration, polymer/solvent ratio, and miscibility).
(polymer degradation (controlled by polymer structure e.g., monomers selection, monomer ratios, molecular weight, end groups, and porosity/morphology).
(drug/polymer miscibility and polarity of solvent, and
molecular weight and lipophilicity of drug.

TABLE 2
examples of compositions
Examples of constituents by % wt. in a composition comprising SRF

| | | | SRF (drug & | SRF |
|---|---|---|---|---|
| Example | Drug | Polymer | polymer %) | (solvent %) |
| 1 | Paclitaxel | PLGA 50/50 | 52.1% | NMP 47.8% |
| | (4.3%) | (47.8%) | (polymer + | |
| | | | drug) | |
| 2 | Sirolimus | PLGA 85/15 | 52.1% | NMP 47.8% |
| | (4.3%) | (47.8%) | (polymer + | |
| | | | drug) | |
| 3 | Paclitaxel | PLGA 50/50 | 51.8% | NMP 48.2% |
| | (3.6%) | (48.2%) | (polymer + | |
| | | | drug) | |
| 4 | Sirolimus | PLGA 85/15 | 51.8% | NMP 48.3% |
| | (3.4%) | (48.3%) | (polymer + | |
| | | | drug) | |
| 5 | Paclitaxel | PLGA 50/50 | 52.5% | NMP 47.5% |
| | (5.0%) | (47.5%) | (polymer + | |
| | | | drug) | |
| 6 | Sirolimus | PLGA 85/15 | 52.5% | NMP 47.5% |
| | (5.0%) | (47.5%) | (polymer + | |
| | | | drug) | |
| 7 | Sirolimus | PLGA 50/50 | 53.5% | NMP 46.5% |
| | (7.0%) | (46.5%) | (polymer + | |
| | | | drug) | |
| 8 | Sirolimus | PLGA 85/15 | 53.5% | NMP 46.5% |
| | (7.0%) | (46.5%) | (polymer + | |
| | | | drug) | |
| 9 | Paclitaxel | PLGA 50/50 | 53.5% | NMP 46.5% |
| | (7.0%) | (46.5%) | (polymer + | |
| | | | drug) | |
| 10 | Paclitaxel | PLGA 85/15 | 53.5% | NMP 46.5% |
| | (7.0%) | (46.5%) | (polymer + | |
| | | | drug) | |
| 11 | Sirolimus | PLGA 50/50 | 45.5% | NMP 54.5% |
| | (8.0%) | (37.5%) | (polymer + | |
| | | | drug) | |
| 12 | Sirolimus | PLGA 85/15 | 45.5% | NMP 54.5% |
| | (8.0%) | (37.5%) | (polymer + | |
| | | | drug) | |
| 13 | Paclitaxel | PLGA 50/50 | 45.5% | NMP 54.5% |
| | (8.0%) | (37.5%) | (polymer + | |
| | | | drug) | |
| 14 | Paclitaxel | PLGA 85/15 | 45.5% | NMP 54.5% |
| | (8.0%) | (37.5%) | (polymer + | |
| | | | drug) | |
| 15 | Sirolimus | PLGA 50/50 | 49.0% | NMP 51.0% |
| | (9.0%) | (40.0%) | (polymer + | |
| | | | drug) | |
| 16 | Sirolimus | PLGA 85/15 | 49.0% | NMP 51.0% |
| | (9.0%) | (40.0%) | (polymer + | |
| | | | drug) | |
| 17 | Paclitaxel | PLGA 50/50 | 49.0% | NMP 51.0% |
| | (9.0.%) | (40.0%) | (polymer + | |
| | | | drug) | |
| 18 | Paclitaxel | PLGA 85/15 | 49.0% | NMP 51.0% |
| | (9.0%) | (40.0%) | (polymer + | |
| | | | drug) | |
| 19 | Sirolimus | PLGA 50/50 | 50.0% | NMP 50.0% |
| | (10.0%) | (40.0%) | (polymer + | |
| | | | drug) | |
| 20 | Sirolimus | PLGA 85/15 | 50.0% | NMP 50.0% |
| | (10.0%) | (40.0%) | (polymer + | |
| | | | drug) | |
| 21 | Paclitaxel | PLGA 50/50 | 50.0% | NMP 50.0% |
| | (10.0%) | (40.0%) | (polymer + | |
| | | | drug) | |
| 22 | Paclitaxel | PLGA 85/15 | 50.0% | NMP 50.0% |
| | (10.0%) | (40.0%) | (polymer + | |
| | | | drug) | |
| 23 | Sirolimus | PLGA 50/50 | 47.0% | NMP 53.0% |
| | (12.0%) | (35.0%) | (polymer + | |
| | | | drug) | |
| 24 | Sirolimus | PLGA 85/15 | 47.0% | NMP 53.0% |
| | (12.0%) | (35.0%) | (polymer + | |
| | | | drug) | |
| 25 | Paclitaxel | PLGA 50/50 | 47.0% | NMP 53.0% |
| | (12.0%) | (35.0%) | (polymer + | |
| | | | drug) | |
| 26 | Paclitaxel | PLGA 85/15 | 47.0% | NMP 53.0% |
| | (12.0%) | (35.0%) | (polymer + | |
| | | | drug) | |
| 27 | Docetaxel | PLGA 50/50 | 45.5% | NMP 54.5% |
| | (8.0%) | (37.5%) | (polymer + | |
| | | | drug) | |
| 28 | Docetaxel | PLGA 85/15 | 45.5% | NMP 54.5% |
| | (8.0%) | (37.5%) | (polymer + | |
| | | | drug) | |
| 29 | Docetaxel | PLGA 50/50 | 49.0% | NMP 51.0% |
| | (9.0%) | (40.0%) | (polymer + | |
| | | | drug) | |
| 30 | Docetaxel | PLGA 85/15 | 49.0% | NMP 51.0% |
| | (9.0%) | (40.0%) | (polymer + | |
| | | | drug) | |
| 31 | Docetaxel | PLGA 50/50 | 49.0% | NMP 51.0% |
| | (9.0.%) | (40.0%) | (polymer + | |
| | | | drug) | |
| 32 | Docetaxel | PLGA 85/15 | 49.0% | NMP 51.0% |
| | (9.0%) | (40.0%) | (polymer + | |
| | | | drug) | |
| 33 | Docetaxel | PLGA 50/50 | 50.0% | NMP 50.0% |
| | (10.0%) | (40.0%) | (polymer + | |
| | | | drug) | |
| 34 | Docetaxel | PLGA 85/15 | 50.0% | NMP 50.0% |
| | (10.0%) | (40.0%) | (polymer + | |
| | | | drug) | |
| 35 | Docetaxel | PLGA 50/50 | 50.0% | NMP 50.0% |
| | (10.0%) | (40.0%) | (polymer + | |
| | | | drug) | |
| 36 | Docetaxel | PLGA 85/15 | 50.0% | NMP 50.0% |
| | (10.0%) | (40.0%) | (polymer + | |
| | | | drug) | |
| 37 | Docetaxel | PLGA 50/50 | 47.0% | NMP 53.0% |
| | (12.0%) | (35.0%) | (polymer + | |
| | | | drug) | |
| 38 | Docetaxel | PLGA 85/15 | 47.0% | NMP 53.0% |
| | (12.0%) | (35.0%) | (polymer + | |
| | | | drug) | |
| 39 | Docetaxel | PLGA 50/50 | 47.0% | NMP 53.0% |
| | (12.0%) | (35.0%) | (polymer + | |
| | | | drug) | |
| 40 | Docetaxel | PLGA 85/15 | 47.0% | NMP 53.0% |
| | (12.0%) | (35.0%) | (polymer + | |
| | | | drug) | |
| 41 | Everolimus | PLGA 50/50 | 45.5% | NMP 54.5% |
| | (8.0%) | (37.5%) | (polymer + | |
| | | | drug) | |
| 42 | Everolimus | PLGA 85/15 | 45.5% | NMP 54.5% |
| | (8.0%) | (37.5%) | (polymer + | |
| | | | drug) | |
| 43 | Everolimus | PLGA 50/50 | 49.0% | NMP 51.0% |
| | (9.0%) | (40.0%) | (polymer + | |
| | | | drug) | |
| 44 | Everolimus | PLGA 85/15 | 49.0% | NMP 51.0% |
| | (9.0%) | (40.0%) | (polymer + | |
| | | | drug) | |
| 45 | Everolimus | PLGA 50/50 | 49.0% | NMP 51.0% |
| | (9.0.%) | (40.0%) | (polymer + | |
| | | | drug) | |
| 46 | Everolimus | PLGA 85/15 | 49.0% | NMP 51.0% |
| | (9.0%) | (40.0%) | (polymer + | |
| | | | drug) | |
| 47 | Everolimus | PLGA 50/50 | 50.0% | NMP 50.0% |
| | (10.0%) | (40.0%) | (polymer + | |
| | | | drug) | |
| 48 | Everolimus | PLGA 85/15 | 50.0% | NMP 50.0% |
| | (10.0%) | (40.0%) | (polymer + | |
| | | | drug) | |
| 49 | Everolimus | PLGA 50/50 | 50.0% | NMP 50.0% |
| | (10.0%) | (40.0%) | (polymer + | |
| | | | drug) | |
| 50 | Everolimus | PLGA 85/15 | 50.0% | NMP 50.0% |
| | (10.0%) | (40.0%) | (polymer + | |
| | | | drug) | |
| 51 | Docetaxel | PLGA 50/50 | 47.0% | NMP 53.0% |
| | (12.0%) | (35.0%) | (polymer + | |
| | | | drug) | |
| 52 | Docetaxel | PLGA 85/15 | 47.0% | NMP 53.0% |
| | (12.0%) | (35.0%) | (polymer + | |
| | | | drug) | |
| 53 | Carmustine | PLGA 50/50 | 45.5% | NMP 54.5% |
| | (8.0%) | (37.5%) | (polymer + | |
| | | | drug) | |
| 54 | Carmustine | PLGA 85/15 | 45.5% | NMP 54.5% |
| | (8.0%) | (37.5%) | (polymer + | |
| | | | drug) | |
| 55 | Carmustine | PLGA 50/50 | 49.0% | NMP 51.0% |
| | (9.0%) | (40.0%) | (polymer + | |
| | | | drug) | |
| 56 | Carmustine | PLGA 85/15 | 49.0% | NMP 51.0% |
| | (9.0%) | (40.0%) | (polymer + | |
| | | | drug) | |
| 57 | Carmustine | PLGA 50/50 | 49.0% | NMP 51.0% |
| | (9.0.%) | (40.0%) | (polymer + | |
| | | | drug) | |
| 58 | Carmustine | PLGA 85/15 | 49.0% | NMP 51.0% |
| | (9.0%) | (40.0%) | (polymer + | |
| | | | drug) | |
| 59 | Carmustine | PLGA 50/50 | 50.0% | NMP 50.0% |
| | (10.0%) | (40.0%) | (polymer + | |
| | | | drug) | |
| 60 | Carmustine | PLGA 85/15 | 50.0% | NMP 50.0% |
| | (10.0%) | (40.0%) | (polymer + | |
| | | | drug) | |
| 61 | Carmustine | PLGA 50/50 | 50.0% | NMP 50.0% |
| | (10.0%) | (40.0%) | (polymer + | |
| | | | drug) | |
| 62 | Carmustine | PLGA 85/15 | 50.0% | NMP 50.0% |
| | (10.0%) | (40.0%) | (polymer + | |
| | | | drug) | |
| 63 | Carmustine | PLGA 50/50 | 47.0% | NMP 53.0% |
| | (12.0%) | (35.0%) | (polymer + | |
| | | | drug) | |
| 64 | Carmustine | PLGA 85/15 | 47.0% | NMP 53.0% |
| | (12.0%) | (35.0%) | (polymer + | |
| | | | drug) | |
| 65 | Mitomycin | PLGA 50/50 | 45.5% | NMP 54.5% |
| | (8.0%) | (37.5%) | (polymer + | |
| | | | drug) | |
| 66 | Mitomycin | PLGA 85/15 | 45.5% | NMP 54.5% |
| | (8.0%) | (37.5%) | (polymer + | |
| | | | drug) | |
| 67 | Mitomycin | PLGA 50/50 | 49.0% | NMP 51.0% |
| | (9.0%) | (40.0%) | (polymer + | |
| | | | drug) | |
| 68 | Mitomycin | PLGA 85/15 | 49.0% | NMP 51.0% |
| | (9.0%) | (40.0%) | (polymer + | |
| | | | drug) | |
| 69 | Mitomycin | PLGA 50/50 | 49.0% | NMP 51.0% |
| | (9.0.%) | (40.0%) | (polymer + | |
| | | | drug) | |
| 70 | Mitomycin | PLGA 85/15 | 49.0% | NMP 51.0% |
| | (9.0%) | (40.0%) | (polymer + | |
| | | | drug) | |
| 71 | Mitomycin | PLGA 50/50 | 50.0% | NMP 50.0% |
| | (10.0%) | (40.0%) | (polymer + | |
| | | | drug) | |
| 72 | Mitomycin | PLGA 85/15 | 50.0% | NMP 50.0% |
| | (10.0%) | (40.0%) | (polymer + | |
| | | | drug) | |
| 73 | Mitomycin | PLGA 50/50 | 50.0% | NMP 50.0% |
| | (10.0%) | (40.0%) | (polymer + | |
| | | | drug) | |
| 74 | Mitomycin | PLGA 85/15 | 50.0% | NMP 50.0% |
| | (10.0%) | (40.0%) | (polymer + | |
| | | | drug) | |
| 75 | Mitomycin | PLGA 50/50 | 47.0% | NMP 53.0% |
| | (12.0%) | (35.0%) | (polymer + | |
| | | | drug) | |
| 76 | Mitomycin | PLGA 85/15 | 47.0% | NMP 53.0% |
| | (12.0%) | (35.0%) | (polymer + | |
| | | | drug) | |
| 77 | Tacrolimus | PLGA 50/50 | 45.5% | NMP 54.5% |
| | (8.0%) | (37.5%) | (polymer + | |
| | | | drug) | |
| 78 | Tacrolimus | PLGA 85/15 | 45.5% | NMP 54.5% |
| | (8.0%) | (37.5%) | (polymer + | |
| | | | drug) | |
| 79 | Tacrolimus | PLGA 50/50 | 49.0% | NMP 51.0% |
| | (9.0%) | (40.0%) | (polymer + | |
| | | | drug) | |
| 80 | Tacrolimus | PLGA 85/15 | 49.0% | NMP 51.0% |
| | (9.0%) | (40.0%) | (polymer + | |
| | | | drug) | |
| 81 | Tacrolimus | PLGA 50/50 | 49.0% | NMP 51.0% |
| | (9.0.%) | (40.0%) | (polymer + | |
| | | | drug) | |
| 82 | Tacrolimus | PLGA 85/15 | 49.0% | NMP 51.0% |
| | (9.0%) | (40.0%) | (polymer + | |
| | | | drug) | |
| 85 | Tacrolimus | PLGA 50/50 | 50.0% | NMP 50.0% |
| | (10.0%) | (40.0%) | (polymer + | |
| | | | drug) | |
| 86 | Tacrolimus | PLGA 85/15 | 50.0% | NMP 50.0% |
| | (10.0%) | (40.0%) | (polymer + | |
| | | | drug) | |
| 87 | Tacrolimus | PLGA 50/50 | 50.0% | NMP 50.0% |
| | (10.0%) | (40.0%) | (polymer + | |
| | | | drug) | |
| 88 | Tacrolimus | PLGA 85/15 | 50.0% | NMP 50.0% |
| | (10.0%) | (40.0%) | (polymer + | |
| | | | drug) | |
| 89 | Tacrolimus | PLGA 50/50 | 47.0% | NMP 53.0% |
| | (12.0%) | (35.0%) | (polymer + | |
| | | | drug) | |
| 90 | Tacrolimus | PLGA 85/15 | 47.0% | NMP 53.0% |
| | (12.0%) | (35.0%) | (polymer + | |
| | | | drug) | |
| 91 | Temsirolimus | PLGA 50/50 | 45.5% | NMP 54.5% |
| | (8.0%) | (37.5%) | (polymer + | |
| | | | drug) | |
| 92 | Temsirolimus | PLGA 85/15 | 45.5% | NMP 54.5% |
| | (8.0%) | (37.5%) | (polymer + | |
| | | | drug) | |
| 93 | Temsirolimus | PLGA 50/50 | 49.0% | NMP 51.0% |
| | (9.0%) | (40.0%) | (polymer + | |
| | | | drug) | |
| 94 | Temsirolimus | PLGA 85/15 | 49.0% | NMP 51.0% |
| | (9.0%) | (40.0%) | (polymer + | |
| | | | drug) | |
| 95 | Temsirolimus | PLGA 50/50 | 49.0% | NMP 51.0% |
| | (9.0.%) | (40.0%) | (polymer + | |
| | | | drug) | |
| 96 | Temsirolimus | PLGA 85/15 | 49.0% | NMP 51.0% |
| | (9.0%) | (40.0%) | (polymer + | |
| | | | drug) | |
| 97 | Temsirolimus | PLGA 50/50 | 50.0% | NMP 50.0% |
| | (10.0%) | (40.0%) | (polymer + | |
| | | | drug) | |
| 98 | Temsirolimus | PLGA 85/15 | 50.0% | NMP 50.0% |
| | (10.0%) | (40.0%) | (polymer + | |
| | | | drug) | |
| 99 | Temsirolimus | PLGA 50/50 | 50.0% | NMP 50.0% |
| | (10.0%) | (40.0%) | (polymer + | |
| | | | drug) | |
| 100 | Temsirolimus | PLGA 85/15 | 50.0% | NMP 50.0% |
| | (10.0%) | (40.0%) | (polymer + | |
| | | | drug) | |
| 101 | Temsirolimus | PLGA 50/50 | 47.0% | NMP 53.0% |
| | (12.0%) | (35.0%) | (polymer + | |
| | | | drug) | |
| 102 | Temsirolimus | PLGA 85/15 | 47.0% | NMP 53.0% |
| | (12.0%) | (35.0%) | (polymer + | |
| | | | drug) | |

The composition may include a bioabsorbable polymer at a concentration of 20-80%, 25-75%, 35-60% by wt. of the bioabsorbable polymer composition, 80-20%, 75-25%, 60-40%, by wt. of the solvent and 0.5%-30% by wt. drug; 1%-20% by wt. of drug, or 1%-5% by wt. of drug.

The composition is delivered in a minimal manner, minimized between 10-200 microliter per injection over 1-5, or 1-10 injections across fibroids or regions of the uterus to reduce fibroid tissue pressure on the urethra. This should mitigate any potential backflow through the delivery device and/or loss of therapeutic injectate from the target tissue. Alternatively, an injection volume range 50-100 microliter per injection over 1-5 injections across each fibroid region of the uterus can be done. The low unit volume injections are designed to minimize swelling and bulking of the uterus. Total volumes includes the amount of unit volumes delivered to each size fibroid across one or multiple fibroids of a uterus.

In addition to the search for a composition that could satisfy the first two of the three characteristics sought by the inventors (minimal uncontrolled drug diffusion, sustained release rate and efficacy), there was the concomitant need to determine what kind of needle syringe could deliver the total volume of composition effectively, in a repeatable manner as one or more low unit volumes without the need for compromising the effectiveness of the SRF once injected. Thus, investigation into the deliverability of the composition to a patient was also needed.

A long, narrow needle (e.g., 25 cm with 0.06 cm lumen size) is desired as the manner for delivering the composition to the target tissue. Because it is a less invasive approach to accessing the target tissue than other methods for treating fibroids and is more likely to be acceptable by patients in general. But use of this type of delivery device presented special challenges that needed to be overcome before the three characteristics of uterus treatment could be satisfied when using this delivery device.

Delivery of a SRF, a composition characterized by a relatively high viscosity for dispensing from a needle, is challenging when using a long, narrow needle of high slenderness and when dispensed in small volumes, due to the liquid shear stresses influenced by the needle inner surface area. The resistance to flow by a composition pushed through a long and narrow needle becomes more acute and deserving of attention the more viscous the liquid. Accordingly, for a relatively high viscosity fluid, which is more influenced by wall-fluid shearing stresses than a less viscous fluid, the inaccuracy in volume dispensed from a needle is more pronounced and varies proportionately with needle slenderness (defined as needle length, L, divided by lumen diameter, D). Accordingly, since the inaccuracy of volume delivered varies proportionately with L/D and directly with the composition's viscosity, the smaller the dispensed volume desired and more viscous the composition, the more inaccuracies are expected. The inventors, realizing this challenge to overcome with accurately dispensing the viscous composition for uterus treatment, had to identify both the embodiments of composition satisfying the first and second characteristics (minimal uncontrolled drug diffusion, sustained release rate and efficacy requirements) and the third characteristic (deliverability): the needle syringe(s) that when used by a health professional enabled him/her to deliver a low unit volume distributed over the uterus to attain the total volume of composition, without unduly compromising SRF effectiveness when introduced into the uterus.

On the one hand, bench tests indicated promising SRF formulations, but the corresponding compositions could not be reliably delivered using a long narrow needle. On the other hand, when the viscosity of the composition was reduced to make it easier to dispense the composition at the desired unit volume, either the desired drug release profile and/or gelation rate would not be achieved, or there would be an unacceptable level of drug diffusion into adjacent tissue or organs. Either the drug eluted too quickly from the SRF, the polymer gel was not dense or entangled enough, or the gelation rate was not high enough to avoid injectate flowing back to the needle and being drawn back along with the needle, away from the target tissue, after completion of the injection.

Thus, a careful balancing of constituents in the formulation—a polymer structure and molecular weight and concentration with a drug ratio and dose and solvent ratio to produce a desirable SRF with controlled release (e.g., as shown in FIG. 4, TABLE 4A)—was eventually accomplished. But then it was found that the desired composition could not be delivered reliably as several low unit volumes using a long needle as required to treat the uterus. Not infrequently, a more preferred, viscous formulation would need to be abandoned for a less viscous formulation to ensure that the right unit volume of composition with desired drug release could be repeatedly delivered using a long and narrow needle. Furthermore, for fibroid treatment there is the additional requirement of sufficient external coverage and penetration.

As for compositions comprising the SRF, drug and polymer concentrations in the composition may range from 0.1 wt % up to 60 wt %. Injection volumes of the SRF may range from 25 microliters up to 5 mL per injection. Overall dosage of drug provided in the SRF can range from 50 ug up to 200 mg. For example, a composition comprises 300 ug of sirolimus dissolved with 3,000 ug (“ug”-micrograms) poly (D, L-lactide-co-glycolide) (85:15) in NMP at an about 50 solid wt. % concentration, a composition, a composition comprises 300 ug of paclitaxel dissolved with 3,000 ug (“ug”—micrograms) poly (D, L-lactide-co-glycolide) (85:15) in NMP at an about 50 solid wt. % concentration, a composition comprises 500 ug of sirolimus dissolved with 2,900 ug (“ug”—micrograms) poly (D, L-lactide-co-glycolide) (85:15) in NMP at an about 50 solid wt. % concentration, a composition comprises 500 ug of paclitaxel dissolved with 2,900 ug (“ug”—micrograms) poly (D, L-lactide-co-glycolide) (85:15) in NMP at an about 50 solid wt. % concentration, a composition comprises 300 ug of sirolimus dissolved with 3,000 ug (“ug”—micrograms) poly (D, L-lactide-co-glycolide) (50:50) in NMP at an about 50 solid wt. % concentration, a composition, a composition comprises 300 ug of paclitaxel dissolved with 3,000 ug (“ug”—micrograms) poly (D, L-lactide-co-glycolide) (50:50) in NMP at an about 50 solid wt. % concentration, a composition comprises 500 ug of sirolimus dissolved with 2,900 ug (“ug”—micrograms) poly (D, L-lactide-co-glycolide) (50:50) in NMP at an about 50 solid wt. % concentration, and a composition comprises 500 ug of paclitaxel dissolved with 2,900 ug (“ug”—micrograms) poly (D, L-lactide-co-glycolide) (50:50) in NMP at an about 50 solid wt. % concentration, the term solvent refers to a solvent capable of fully or homogenously dissolving substances added to the solvent.

Further examples of compositions that satisfy the first and second characteristic (efficacy and sustained release, low diffusion) are provided in the examples below.

Precise delivery of low volumes of a SRF for therapeutic retention with a precise drug dosage may be accomplished using a 250-500, or 250-2500 microliter or smaller needle syringe. This delivery device may have a gastight, glass cyclic olefin polymer, or polypropylene syringe body with Teflon, polyethylene, cyclic olefin polymer, or polypropylene plunger connected with a 20 cm long 20 gG needle, which has a 0.06 cm lumen diameter. The volume of the delivery syringe must be large enough to accommodate the dead volume within the needle of approximately 150 microliter. Therefore, a 250 microliter gastight syringe can be utilized to deliver one to two 50 microliter unit volumes of a composition. For multiple injections from the same delivery device a 250-1,000 microliter gastight syringe volume may be used, which allows sufficient volume to overcome needle dead volume and can deliver at least eight 100 microliter unit volume injections to each fibroid.

Alternatively, a 1,000 microliter or 1 milliliter cyclic olefin polymer and/or polypropylene syringes may be utilized. For example, the SRF may be provided as a kit containing one syringe with the PLGA dissolved in NMP solvent with female luer lock and the other syringe with the drug powder with male luer lock. These syringes are then directly connected and mixed back and forth a number of times (fifty) to completely solubilize the drug such that no drug particles remain. A lower number of mixes (ten) can also be utilized to result in a partially mixed SRF with remaining drug particulate that can produce an extended drug release time in vivo due to the additional requirement of drug particle dissolution in the SRF. After mixing the SRF is loaded into the graduated syringe for delivery via long slender needle.

Precise delivery of low volumes of a SRF for therapeutic injection with a precise drug dosage may also be accomplished using a 2,000 microliter or 2 milliliter syringe. This delivery device may have a gastight, glass cyclic olefin polymer, or polypropylene syringe body with Teflon, polyethylene, cyclic olefin polymer, or polypropylene plunger connected with a 45 cm long 23 g needle, which has a 0.03 cm lumen diameter. The volume of the delivery syringe must be large enough to accommodate the dead volume within the needle of approximately 1.26 milliliter. Therefore, a 1,500 microliter gastight syringe can be utilized to deliver one to two 50 microliter unit volumes of a composition. For multiple injections from the same delivery device a 2,000-3,000 microliter gastight syringe volume may be used, which allows sufficient volume to overcome needle dead volume and can deliver at least eight 100 microliter unit volume injections to a fibroid. Alternatively, a 3,000 microliter or 3 milliliter cyclic olefin polymer and/or polypropylene syringes may be utilized. For example, the SRF may be provided as a kit containing one syringe with the PLGA dissolved in NMP solvent with female luer lock and the other syringe with the drug powder with male luer lock. These syringes are then directly connected and mixed back and forth a number of times (fifty) to completely solubilize the drug such that no drug particles remain. A lower number of mixes (ten) can also be utilized to result in a partially mixed SRF with remaining drug particulate that can produce an extended drug release time in vivo due to the additional requirement of drug particle dissolution in the SRF. After mixing the SRF is loaded into the graduated syringe for delivery via long slender needle.

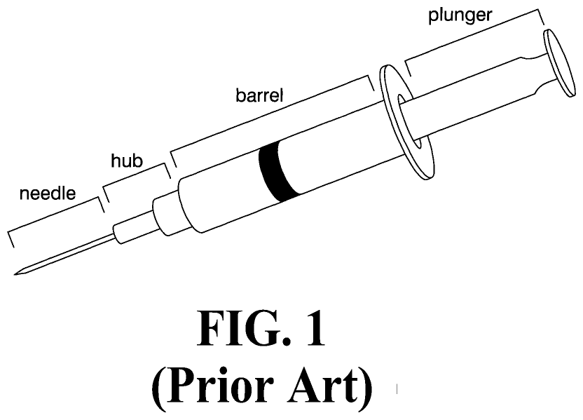

Referring to FIG. 1, a typical needle syringe includes an applicator portion (plunger, barrel), and hub portion that connects the needle with the interior chamber of the barrel and forms a fluid-tight connection for passage of a fluid contained within the barrel through the needle lumen.

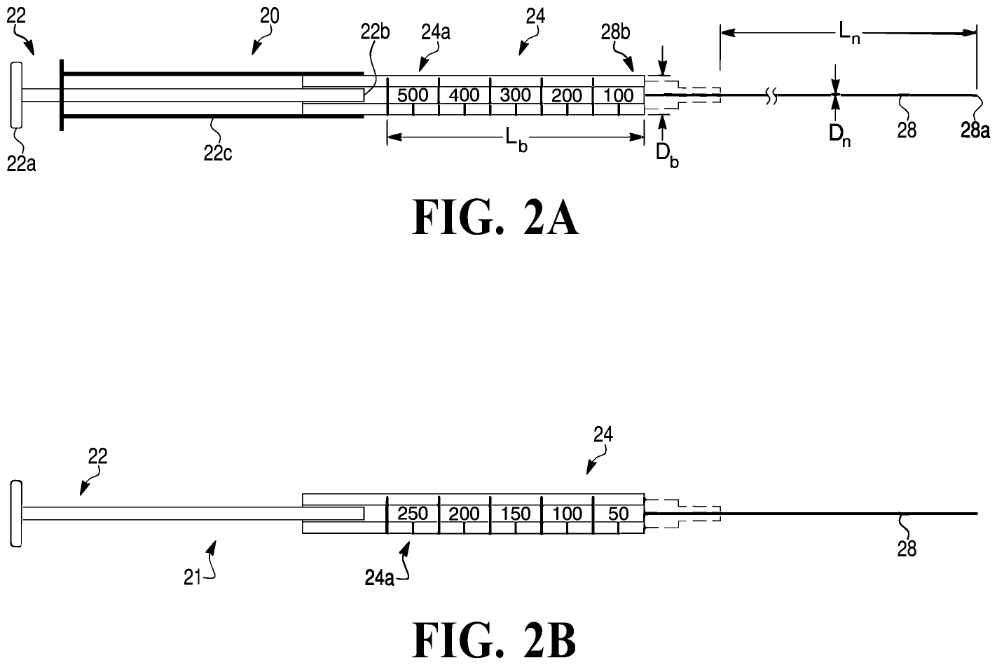

Referring to FIGS. 2A and 2B, there is shown side view schematics of needle syringes 20, 21 according to the disclosure. More specifically, the needle syringes depicted have sizes for holding and dispensing a unit volume through a long, narrow needle tip 28a. The needle 28 is characterized by a length Ln and lumen diameter Dn (a needle hub is shown in phantom for the sake of illustrating the measured length Ln, which is taken from the upstream opening 28b forming the fluid-tight connection between the barrel and needle 28 body and the terminal, or downstream-most tip that first punctures the skin). The needle may be made from stainless steel.

The needle syringe in FIG. 2A shows by example gradations of 500/400/300/200/100 micro-liters for dispensing unit volumes of e.g., 0.05 ml (50 micro-liters). The barrel capacity is 500 micro-liters. The barrel 24 has a length Lb and barrel inner or lumen diameter Db. The prefilled syringe barrel (500 micro-liter) may have attached to it a needle 28 having a length Ln of 20 cm and the barrel may have a 150-200 micro-liter dead volume. The syringe which includes a proximal syringe plunger body 22 and which may need to be prefilled, may also incorporate a stabilizer 22c for guiding the plunger head 22b towards the needle opening 28b. The composition comprising the SRF (contained in the syringe barrel 24 and in amounts up to about 500 micro-liters) is ejected through the needle tip 28a by finger pressure applied to the plunger head 22a. In this example, the barrel, when holding 500 micro-liters of composition can hold, for example, up to 7, 50 micro-liter unit volumes accounting for the 150 micro-liter dead volume. The needle syringe in FIG. 2B is the same in several respects as the syringe in FIG. 2A, except that it has instead a 3000 micro-liter barrel capacity. In another example a 3000 micro-liter capacity syringe has graduations of 200 uL, 100 uL, and 50 uL to help with more accurately dispensing low unit volumes of the composition.

In some cases, a SRF delivery dosing guide can be included with the needle syringe to accurately dispense the unit volume. This dosing guide is attached to the proximal syringe plunger body 22 exposed outside of the needle. When attached the dosing guide will only enable syringe plunger translation to deliver the precise unit volume of e.g., 0.05 mL (50 micro-liters). The travel distance will be calibrated based in the inner diameter of the barrel. For example, a 1 mL syringe barrel has an inner diameter of 5 millimeters. To dispense a unit volume of 0.05 mL the dosing guide would be calibrated to an allowed plunger displacement of 2.55 mm. The dosing guide can be inserted flush with the proximal body of the syringe barrel while attached to the plunger. After each unit volume injection, it can be removed and re-loaded for the next unit volume.

The syringes in FIGS. 2A and 2B are sized to enable a medical professional to accurately dispense unit volume amounts (e.g., 0.05 ml or 0.1 ml) at discrete/separate locations within a target tissue. The syringe may be pre-filled to hold a sufficient total volume for fully treating a patient (e.g., 4 unit volumes injectate at different locations in the uterus tissue). In some embodiments, an applicator portion may include syringe chambers for holding the SRF drug and drug carrier separate from each other, a mixing element for combining the drug and drug carrier such as a static mixing Y-adapter that feeds into a narrow and long, or high slenderness (L/D) ratio needle 28. Alternatively, the drug, drug carrier, and solvent may be mixed using two syringes connected with an adapter, with back and forth plunging to mix the drug, drug carrier, and polymer. Alternatively, the two syringes may connect directly to mix using mated female and male luer fittings at the distal location of each syringe. The applicator portion may have markings demonstrating measurement of lengths of needle insertion or composition volume.

The syringe control needed for dispensing unit volumes of a composition comprising a SRF from the barrel 22 through the needle tip 28 becomes challenging either when a long narrow needle is used, or individual injections do not exceed a unit volume amount. Both of these things are present according to the disclosure, as explained earlier. It was found that for the ranges of SRF formulations found suitable to achieve the goals of treatment and being deliverable in a composition in unit volume amounts, the following values for Lb, Db, Ln, and Dn can apply. The needle length Ln may be from 15 to 45 cm, more preferably from 25 to 30 cm. The lumen diameter Dn may be 0.06 cm, or between 0.06 and 1.19 cm, and in some embodiments 0.02 cm up to 0.12 cm. The needle's slenderness ratio (Ln/Dn) may be between 125 and 750, or between 400 and 750.

The barrel length Lb may be from 5 to 20 cm, more preferably from 5 to 10 cm. The barrel lumen diameter Db may be 0.2 to 0.5 cm.

In some embodiments, the syringe barrel's slenderness ratio (Lb/Db may be 5 and 50, or between 10 and 30) is an important factor to consider. Barrel slenderness ratios in these ranges were found to help with more accurately dispensing low unit volumes of the composition.

A desired injection volume control and precision custom elongated glass body gastight delivery syringe can be utilized that provides increased accuracy of volume graduation markings on the syringe. Instead of volume graduations these delivery device markings can also show directly the needle calibrated dead volume and drug delivery drug dosage in mg for each injection for the user.

The pre-filled syringe preferably contains the composition as a pre-mixed solution for each injection, which results in delivery of a primarily amorphous drug already dissolved in the polymer carrier for faster drug release and enhanced onset of efficacy then later slower sustained release of drug.

In some embodiments, an SRF can be provided with polymer injectate and drug powder in the separate delivery syringes (mentioned above) mixed by the user on or about the time of treatment and placed into the syringe barrel. This would allow for a mostly or partially crystalline drug directly into the injectate to increase drug retention time and exposure time and reduce systemic drug loss given that some drug would remain in a powdered crystalline form after user preparation.

In some embodiments, An SRF generating composition can be mixed with an ultrasound microbubble contrast agent to further enhance visibility of injectate under ultrasound guidance to enable precise location control of injection within a uterus to further minimize potential for drug loss to the healthy uterus, surrounding organs and systemic circulation.

While testing different formulations for an efficacious SRF, the inventors had sought to find a parameter or relationship that they could use to discount a formulation as unsuitable because it could not be delivered reliably. This was much needed because the iterative process of conceiving a certain formulation then evaluating whether it, in combination with the delivery vehicle could satisfy all three characteristics of fibroid treatment was a long process. To find a formulation that eventually met those requirements, only to find out that it could not be reliably delivered in the small volumes needed using a long narrow needle, made the process much more labor intensive.

Moreover, it should be emphasized that the objective in matching a composition with a needle syringe was to not compromise more than needed, on the effectiveness of the SRF, to ensure its deliverability. The inventors sought an advantageous solution, not simply a way to save time or reduce the amount work needed to match an acceptable SRF with a delivery device. Unexpectedly, it was discovered that there is a numerical range to inform whether a unit volume v of a composition comprising an SRF (as represented by its absolute viscosity p) can be accurately and repeatedly delivered using a needle with length L and lumen diameter D.

When adopting this numerical range as a requirement for the syringe needle with composition, in addition to the required three characteristics for fibroid treatment, a more effective SRF formulation is found, because the value addresses the deliverability problem and without unnecessarily compromising the SRF effectiveness. The numerical range is computed using the H-Injectate Rating or HIR, defined as follows:

$$HIR = \frac{μ {L}^{2}}{vD} ( 1 {0}^{- 6} )$$

For a HIR between 20 and 400, with units of centipoise (cP) per unit area, one may conclude that the SRF can be reliably and consistently delivered in unit volume increments using a needle having a length L, lumen diameter D, a composition's absolute viscosity p and a unit volume v. The HIR is defined for L from 15 to 45 cm, more preferably from 25 to 35 cm, a lumen diameter D of 0.06 cm, or between 0.06 and 0.08 cm, and in some embodiments 0.02 cm up to 0.12 cm. The needle's slenderness ratio (L/D) may be between 125 and 750, or between 250 and 350.

In some embodiments HIR may be between 20 and 1300. The higher ranges may be preferred in situations where a more delayed release rate and/or higher rate of gelation is desired, as well as smaller unit volumes. A higher HIR may also be preferred for a larger uterus where a longer needle is needed to access the uterus transvaginal. In some embodiments HIR may be 10 to 300. A smaller HIR may be preferred for a smaller uterus where a shorter needle is needed to access the uterus, laparoscopically.

TABLE 3 (shown in FIGS. 3A, 3B, 3C) discloses embodiments of an apparatus for delivery of a unit volume of a composition comprising an SRF according to the disclosure.

As explained earlier, it is desirable to use a long, slender needle for injecting the relatively high-viscosity composition comprising a SRF that will produce the efficacious result without concomitant, adverse effects on the patient's health due to diffusion, infection, or pain/discomfort during or after the procedure. And it is also important that the medical professional treating the patient has adequate control over the volume of composition injected to enable the dispensing of a unit volume at each injection site, for volume control is necessary to achieve the desired clinical outcome. The ranges in TABLE 3 for HIR are believed effective in identifying the composition satisfying the first and second HIR characteristics for uterus treatment according to the disclosure, and the syringe needle satisfying the third HIR requirement for uterus treatment according to the disclosure.

The lower end of the HIR range in TABLE 3 represents compositions that while relatively easy to dispense in small unit volumes (for a fixed needle length, e.g., 15 cm), have a minimal amount of viscosity representative of an SRF's ability to satisfy two of the characteristics for uterus treatment (efficacy, sustained release, and minimal uncontrolled drug diffusion), shorter needle lengths and higher unit volumes. The upper end of the HIR range in TABLE 3 represent (for a fixed needle length, e.g., 20 cm) compositions having higher viscosity, longer needle lengths and smaller unit volumes. For these compositions, the two characteristics for uterus treatment will be easily satisfied when the formulation is injected, but the viscosity, if any higher, might challenge the health professional's ability to deliver a total volume of composition in unit volume amounts, especially along a longer needle length. As such, the capabilities of the delivery device are limited for high HIR values.

In addition to HIR, in some embodiments the syringe barrel's slenderness (Lb/Db may be 5 and 50, or between 10 and 30) can be an important factor to consider as well. Barrel slenderness ratios in these ranges were found to help with more accurately dispensing low unit volumes of the composition and may be preferred to satisfy the deliverability requirement (third characteristic).

Following description provides additional detailed information on embodiments of an SRF and the various constituents and properties therefore that may be included in a SRF for treating fibroids using a needle syringe according to the disclosure.

Drug or drug combinations used in the SRF include a cytostatic drug, cytotoxic drug, and/or other drugs. The other drug(s) may be used by themselves (i.e., the “other drug(s)” are the only active agents in the SRF), or in combination with the cytostatic drug or cytotoxic drug as part of the medical procedure for treatment of fibroids. For example, the other drug(s) may be administered before the composition injected into the target tissue using a needle syringe, or the other drug(s) may be included in the composition with the SRF. Or the other drug(s) may be administered after the SRF containing the cytostatic drug or cytotoxic drug is administered to the target tissue.

These other drugs, which may be administered with, or instead of the cytostatic or cytotoxic drug, include hormones, hormone agonists and antagonists, analgesics, antibiotics, stimulants, anesthesia, antidepressants, anti-inflammatory drugs, immunotherapy, ablative agents, contraceptives or antifibrinolytics. Hormones may include gonadotropin or progesterone. Hormone agonists or antagonists may include and are not limited to goserelin, leuprorelin, leuprolide acetate, nafarelin, buserelin, buserelin acetate, histrelin, deslorelin, suprelorin, and triptjorelin, Analgesics may include and are not limited to ibuprofen, acetaminophen, naproxen, aspirin, lidocaine, codeine, fentanyl, hydrocodone, methadone, oxycodone, xylocaine, buvipocaine, bupivacaine, cocaine, ropivacaine, mepivacaine, etidocaine, levobupivacaine, trimecaine, carticaine, articaine, lidocaine, prilocaine, benzocaine, procaine, tetracaine, chloroprocaine, and combinations thereof. Antidepressants may include citalopram, escitalopram, fluoxetine, fluvoxamine, paroxetine, and sertraline. Antibiotics may include and are not limited to amoxicillin, doxycycline, cephalexin, ciprofloxacin, clindamycin, metronidazole, azithromycin, sulfamethoxazole, trimethoprim, clavulanate, and levofloxacin. Stimulants may include but are not limited to amphetamine, methylphenidate, amphetamine, dexmethylphenidate, and atomoxetine hydrochloride. Anti-inflammatory drugs may include but are not limited to corticosteroids such as dexamethasone, fluticasone propionate, triamcinolone acetonide, mometasone furoate, prednisone, hydrocortisone, estradiol, clobetasol, fluticasone furoate, methylprednisolone, ketorolac, and budesonide. Contraceptives may include and are not limited to levonorgestrel, etonogestrel, medroxyprogesterone, ethinyl estradiol, drospirenone, norgestimate, norethindrone. Immunotherapy drug types may block cytokine activity or inhibit binding of cytokines to inhibit inflammatory signals such as anti-IL1, anti-IL 2, anti-IL3, anti-IL4, anti-IL8, anti-IL15, anti-IL 18, anti-MCP 1, anti-CCR2, anti-GM-CSF, anti-TNF antibodies, enzalutamide and others. Antifibrinolytics may include but are not limited to tranexamic acid, aminocaproic acid, and aprotinin.

GnRH agonist therapy reduces the fibroid volume by 50% when given systemically. Minimal reduction occurs in less than six months. An up to 50% reduction in fibroid volume is expected in 6 months upon which therapy is stopped to prevent long term side effects.

When expressing a % of a substance in the SRF, the % of that substance may be expressed in terms of a percent weight of the drug(s) to the overall weight of the SRF (“% X by wt”), or to the overall volume of the SRF (“% X by vol”). Unless stated otherwise the percent dosage % will, by default, always refer to a % by weight to the total measured SRF. Unless stated otherwise, weights are given in grams (“g”) or milligrams (“mg”), molecular weight in kilo-Daltons (“kDa”), volume in microliters (“μL”) or milliliters (“mL”), and viscosity units are expressed as inherent viscosity (i.e., the ratio of the natural logarithm of the relative viscosity to the mass concentration of the substance, such as a polymer. The unit of inherent viscosity is deciliters per gram (dL/g). A different measure of viscosity is intrinsic viscosity, which is a measure of a solute's contribution to the total viscosity. Another viscosity is dynamic viscosity or absolute viscosity, the units of which are centimeter-gram-seconds, also known as centipoise (cP).

The SRF may comprise 0.1-60% or 10-25% of a polymer composition, or more preferably 30-50% of a polymer composition. The SRF may comprise 10% solvent. The SRF may comprise 20% solvent. The SRF may comprise 30% solvent. The SRF may comprise 40% solvent. The SRF may comprise 20-80% solvent. The drug to polymer weight ratio of the SRF may be 1:100, 1:50, 1:25, 1:20, 1:10, 1:5, 1:2, 1:1, 2:1, or 5:1. The SRF, once located at the target tissue, may release 1-10%, or 11-50% of the drug load in less than 24 hours, 24-72 hours, 3-7 days, 1-4 weeks, 1-3 months, or more than 3 months. The SRF may release 80-100% in 24-72 h, 3-7 days, 1-4 weeks, 1-3 months, or more than 3 months.

Polymer for use in the SRF as drug carriers include silk-elastin like protein polymers, Pluronics F68 or F127 or a combination thereof, poly(ε-caprolactone) (PC), polylactides (PLA), poly(D,L-lactide) (PDLA), poly(ortho esters), polyanhydrides, polycarbonates, polyethylene glycol (PEG), polyethylene oxide (PEO), polyesteramides, and any combinations thereof including block and random co-polymers such as but not limited to poly(lactide-co-glycolide) (PLGA) and PLGA-PEG-PLGA. More specifically the PLGA composition may consist of poly(D,L-lactide-co-glycolide) (50:50), poly(D-lactide-co-glycolide) (50:50), poly(L-lactide-co-glycolide) (50:50), poly(D,L-lactide-co-glycolide) (65:35), poly(D-lactide-co-glycolide) (65:35), poly(L-lactide-co-glycolide) (65:35), poly(D,L-lactide-co-glycolide) (75:25), poly(D-lactide-co-glycolide) (75:25), poly(L-lactide-co-glycolide) (75:25), poly(D,L-lactide-co-glycolide) (85:15) or a mixture thereof. The PLGA may be end-capped with ester, acid, alcohol, thiol or other end-groups. The inherent viscosity of the PLGA polymer may vary from 0.1 dL/g to greater than 1.0 dL/g. The molecular weight of the PLGA polymer may vary from 10 kDa up to 150 kDa. The polymer may be linear, branched, hyperbranched, dendritic, have a star structure, or be a dendrimer-like star polymer.

Additional embodiments of the polymer, SRF, and composition follow.

Polymer compositions include poly(lactide-co-glycolide), poly(D,L-lactide-co-glycolide), poly(D,L-lactide), ester end capped poly(D,L-lactide-co-glycolide) (50-50), ester end capped poly(D,L-lactide-co-glycolide) (65-35), ester end capped poly(D,L-lactide-co-glycolide (75-25), ester end capped poly(D,L-lactide-co-glycolide (85-15), acid end capped poly(D,L-lactide-co-glycolide) (50-50), acid end capped poly(D,L-lactide-co-glycolide) (65-35), acid end capped poly(D,L-lactide-co-glycolide (75-25), acid end capped poly(D,L-lactide-co-glycolide (85-15), ester end capped poly(D-lactide-co-glycolide) (50-50), ester end capped poly(D-lactide-co-glycolide) (65-35), ester end capped poly(D-lactide-co-glycolide (75-25), acid end capped poly(D-lactide-co-glycolide) (50-50), acid end capped poly(D-lactide-co-glycolide) (65-35), acid end capped poly(D-lactide-co-glycolide (75-25), ester end capped poly(L-lactide-co-glycolide) (50-50), ester end capped poly(L-lactide-co-glycolide) (65-35), ester end capped poly(L-lactide-co-glycolide (75-25), acid end capped poly(L-lactide-co-glycolide) (50-50), acid end capped poly(L-lactide-co-glycolide) (65-35), acid end capped poly(L-lactide-co-glycolide (75-25), ester end capped poly(D,L-lactide-co-glycolide), acid end capped poly(D,L-lactide-co-glycolide), or combinations thereof.

In some embodiments the drug carrier may include a bioabsorbable polymer composition and the inherent viscosity of the polymer composition is between 0.1-1.0 dL/g, 0.1-0.6 dL/g, or 0.1 to 0.4 dL/g or 0.1 to 0.3 dL/g and the ratio of DL-lactide to glycolide is from 30/70 up to 90/10, 95/5, or 85/15.

In some embodiments the composition may include a bioabsorbable polymer at a concentration of 20-80%, 25-75%, 40-60% by wt. of the bioabsorbable polymer composition, 80-20%, 75-25%, 60-40%, by wt. of the solvent and 0.5%-30% by wt. drug; 1%-20% by wt. of drug, or 1%-10% by wt. of drug.

In an alternative embodiment the SRF may be delivered via injection needle in the form of a solid rod. Extruded, cast or molded rods of polymer carrier containing drug inside (length is about 1 cm long, or less), or a polymer carrier infused with the drug may be delivered in solid rod form by way of injection needle. The unit volume range for the drug contained in, or infused with the polymer may be from 1-20 micro-liter, 40-60% drug by wt., and 60-40% polymer composition.

In some embodiments it may be desirable to formulate the SRF so that the drug carrier is fully biodegraded before the next treatment, e.g., 3 months, 6 months or 12 months after the prior treatment. For example, the polymer would have a ratio of glycolide to lactide of 70:30 up to 15:85 for a less hydrophobic structure (faster degradation) and/or an inherent viscosity less than about 1.0 dL/g or more preferably less than 0.3 dL/g.

A polymer composition, when forming a constituent of the SRF, is a polymer composition that enables or achieves a desired “sustained release” of the one or more drugs to the target tissue. In some embodiments, the polymer composition enables or achieves at least 50%, or up to about 100%, or substantially all drug release between 30 and 90 days, through a combination of diffusion and degradation. In other embodiments up 100% of drug release occurs from 90 to 120 days from treatment. Preferably, there is an initial burst (e.g., up to 50% of drug) followed by a substantially reduced rate of release over the next following month, or several following months following treatment. For example, the drug has a release rate of between 5% to 50% during the first 24 hours from injecting the composition into the uterus and the drug has a release rate of no more than 10% to 75% over the first month, 25% to 95% over the first three months, and/or 50% to 100% over the first six months.

As discussed earlier, the SRF must keep drug exposure to the surrounding fibroid and minimize leak or flow to the health uterus other surrounding organs. The SRF delivery should provide sufficient coverage to the external proliferating fibroid. The SRF has advantages over thermal treatments and other solid implants given the SRF's improved exterior fibroid coverage and ability to flow-penetrate the fibroid. In addition, the SRF should not take up too much volume in the uterus. A desired shape of the drug release curve could be a burst of drug and early tissue exposure for fast efficacy and then reduced rate of drug release over a 3-6 month period for sustained efficacy. In some examples the drug release curve could be a burst of drug and early tissue exposure for fast efficacy and then reduced rate of drug release over a 1-4 month period for sustained efficacy. For example, a fast burst release rate followed by a reduced rate was achieved with N-methyl pyrrolidone (NMP) solvent, which is water soluble. This formulation can provide a high release within the first 24 or 48 hours, or within the first 1 week, two weeks, or up to 3 months, followed by a reduced rate of release.

FIG. 4 shows a Drug Release Curve for Sustained Release Formulation (SRFs) A, B and C. These exemplary SRFs provide a burst followed by gradual release of drug over a period of up to 6 months. SRF A releases about 25% of the drug within the first month, followed by a slow, gradual release where about 95% is released at six months. SRF B has an initial burst of about 50% within the first month, followed by about 100% release at two months. SRF C has an initial burst of about 75% within the first month, with about 100% release at about two months.

TABLE 4A below shows examples of SRF formulations for each of SRF A, B and C. Examples A1, A2 exhibit approximately the same release rate characteristics as SRF A in FIG. 4. Examples B1, B2 exhibit approximately the same release rate characteristics as SRF B in FIG. 4. Examples C1, C2 exhibit approximately the same release rate characteristics as SRF C in FIG. 4. TABLE 4B shows examples of drug released over time.

TABLE 4A
Species of SRF formulations for SRF A, B and C in FIG. 4

| SRF | polymer | drug | % by wt. drug v. polymer | Solvent |
|---|---|---|---|---|
| A1 | PLGA8515 | Sirolimus | 1-2% drug, 50% polymer | NMP |
| A2 | PLGA8515 | Paclitaxel | 1-2% drug, 50% polymer | NMP |
| B1 | PLGA7525 | Sirolimus | 3-5% drug, 45-47% | NMP |
| | | | polymer | |
| B2 | PLGA7525 | Paclitaxel | 3-5% drug, 45-47% | NMP |
| | | | polymer | |
| C1 | PLGA5050 | Sirolimus | 1-5% drug, 45-55% | NMP |
| | | | polymer | |
| C2 | PLGA5050 | Paclitaxel | 1-5% drug, 45-55% | NMP |
| | | | polymer | |

TABLE 4B
plotted ranges for SRF A, B & C (FIG. 4)

| Time | SRF A | SRF B | SRF C |
|---|---|---|---|
| (months) | (drug release %) | (drug release %) | (drug release %) |
| 0 | 0 | 0 | 0 |
| 0.5 | 30 ± 20 | 50 ± 25 | 75 ± 25 |
| 1 | 50 ± 20 | 75 ± 15 | 95 ± 5 |
| 2 | 60 ± 15 | 95 ± 5 | 100 |
| 3 | 70 ± 10 | 100 | |
| 6 | 95 ± 5 | | |

The SRF is delivered to the target tissue in the form of a composition in liquid form (i.e., the SRF's drug(s) and drug carrier are in solution, or in suspension in a solvent when in the delivery vehicle) that gels upon contact with water at the target tissue. The SRF may be made by dissolving the SRF components in a suitable solvent. Suitable solvents for these embodiments include water, N-methylpyrrolidone (NMP), dimethyl sulfoxide (DMSO), 2-pyrrolidone, propylene carbonate, caprolactam, triacetin, alcohols, benzyl benzoate, ethyl acetate, triethyl citrate, benzyl alcohol, glyme (dimethoxyethane), diglyme, and other glycol ethers, and dichloromethane, or any mixture thereof.

As explained earlier, the composition should be formulated at a concentration and viscosity that permits passage through a long, narrow needle having a slenderness ratio within the prescribed ranges deliverable in low unit volumes, and controls the drug release, and drug distribution. Drug to polymer ratios, which influence the viscosity of the composition, may vary from 0.025 to 2.0. Drug and polymer concentrations in solvent may range from 0.1 wt % up to 60 wt % or more preferably 30 wt % to 50 wt %, based on the total weight of the SRF. Unit volumes may range from 0.025 mL up to 0.15 mL per injection or more preferably from 0.05 mL to 0.10 mL per injection. Total volume of the composition for a treatment may range from 0.050 mL up to 1.5 mL or more preferably 0.10 mL to 1.0 mL. Overall dosage of drug provided in the SRF can range from 50 ug up to 200 mg or more preferably from 5 mg to 50 mg. For example, a composition containing the SRF is 300 ug of sirolimus or paclitaxel dissolved with 3000 ug (“ug”—micrograms) poly (D, L-lactide-co-glycolide) (85:15) in NMP at a 50 wt % concentration.

As discussed above, the local drug delivery device is a syringe needle for delivery of the composition including the SRF to the target tissue, as earlier shown and described with reference to FIGS. 2A and 2B. In other embodiments, the applicator portion of a delivery vehicle may include syringe chambers for holding the drug and drug carrier separate from each other, a mixing element for combining the drug and drug carrier such as a static mixing Y-adapter that feeds into a narrow and long, or high slenderness ratio needle. Alternatively, the drug and drug carrier may be mixed using two syringes connected with an adapter, with back and forth plunging to mix the drug and drug carrier. The syringe needle may be imaged under ultrasound to visualize the therapy. Echogenicity may be enhanced by dimpling of the applicator needle or coatings. Echogenicity may also be enhanced by incorporation of contrast enhancing agents such as microbubbles, sulfur hexafluoride, octofluoropropane, air, lipid and/or albumin shells. The applicator may have components for attachment to a rectal ultrasound probe. The applicator may have markings demonstrating measurement of lengths of needle insertion or composition volume. The applicator may have a user-friendly handle and plunger to comfortably deliver the needle to the target tissue. An applicator needle size may range from 16G up to 25G. The needle length may range from 10 cm to 30 cm (e.g., 15 cm, 20 cm length) to reach the target tissue from a transrectal or transperineal approach.

As mentioned earlier, while prior methods may show efficacy in reducing fibroids, they either may require a more invasive procedure (vs. localized treatment using the delivery device as disclosed herein, such as the needle used in the animal study), more frequent treatment due to diffusion or more generalized treatment of fibroids raising the possibility of adverse effects because a higher dosage is needed to treat the area while accounting for leakage or diffusion of the drug to other areas. Adverse effects may include diminished urinary or sexual function. It is desired to have an effective treatment targeting only the target tissue and nowhere else (e.g., avoiding the healthy uterus and ovaries) and to perform the procedure in a less invasive manner for patient acceptance.

An effective SRF injectate for treating the uterus according to the disclosure (1) reduces fibroid volume significantly over a 30 and 90 day period, (2) limits substantially all the active agent to the target tissue, and (3) is capable of being delivered as a series of low unit volumes using a needle syringe. As for point (2), it should be mentioned that other known products for treating fibroids by contrast have their active agents disperse significantly. Moreover, the study indicated a significant ratio of uterus 30-day drug concentration to maximum plasma drug concentration of at least about 10,000. As mentioned throughout, it is important to control diffusion of the agent as this can cause adverse effects on nearby tissue and organs. Additionally, in contrast to most prior or existing techniques for treating fibroids, a comparatively small volume of SRF injectate is needed for efficacy. In the example (pre-clinical study) a unit volume of the composition comprising SRF1 was between 10-200 microliter per injection over 1-10 injections across each side of the uterus, or 50-100 microliter per injection over 1-5 injections across each side of the uterus. Prior approaches for treating fibroids seek to maximize injection volumes for increased drug dosage. An SRF however does not require as high a drug dosage, because the drug is maintained in the target tissue.

Following are additional listing of disclosed embodiments:

0.5 mL N-methyl pyrrolidone (NMP) was added to a vial with 0.25 g sirolimus and vortexed until dissolved. 50 microliter of drug solution was added to 2.5 mL of a 50% PLGA8515A (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% PLGA8515A (0.5 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% PLGA8515E (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% PLGA8515E (0.5 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% PLGA8515A (0.7 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% PLGA8515E (0.7 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% PLGA8515A (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% PLGA8515E (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% PLGA8515A (0.5 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% PLGA8515E (0.7 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% PLGA8515E (0.3 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% PLGA8515A (0.5 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% PLGA8515E (0.7 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 200 microliter of drug solution was added to 2.5 mL of a 50% PLGA8515 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% PLGA7525 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% PLGA7525 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 200 microliter of drug solution was added to 2.5 mL of a 50% PLGA7525 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 50 microliter of drug solution was added to 2.5 mL of a 50% PLGA5050 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 100 microliter of drug solution was added to 2.5 mL of a 50% PLGA5050 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g sirolimus and vortexed until dissolved. 200 microliter of drug solution was added to 2.5 mL of a 50% PLGA5050 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA8515A (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA8515E (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA8515A (0.5 dl/g) in NMP solution using syringe to syringe mixing with a female to female luer connector. 250 microliters in a 1 mL syringe was loaded into a 20 G×20 cm Chiba biopsy needle with depth markers. 0.5 mL N-methyl pyrrolidone (NMP) was added to a vial with 0.25 g everolimus and vortexed until dissolved. 50 microliter of drug solution was added to 2.5 mL of a 50% PLGA8515E (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA8515A (0.5 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA8515E (0.7 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA8515E (0.5 dl/g) in NMP solution using syringe to syringe mixing with a female to female luer connector. 800 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA8515A (0.7 dl/g) in NMP solution using syringe to syringe mixing with a female to female luer connector. 800 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA8515A (0.7 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA8515E (0.7 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 100 microliter of drug solution was added to 2.5 mL of a 50% PLGA8515 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA8515 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA7525 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 100 microliter of drug solution was added to 2.5 mL of a 50% PLGA7525 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA7525A (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA7525E (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA7525A (0.5 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA7525E (0.5 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA7525A (0.7 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA7525E (0.7 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA6535E (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA6535A (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA6535E (0.5 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA6535A (0.5 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA6535E (0.5 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA6535A (0.5 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 50 microliter of drug solution was added to 2.5 mL of a 50% PLGA5050 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 100 microliter of drug solution was added to 2.5 mL of a 50% PLGA5050 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g everolimus and vortexed until dissolved. 200 microliter of drug solution was added to 2.5 mL of a 50% PLGA5050 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 50 microliter of drug solution was added to 2.5 mL of a 50% PLGA8515 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA8515A (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA8515E (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA8515A (0.5 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA8515E (0.5 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA8515A (0.7 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA8515E (0.7 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA8515A (0.5 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA8515E (0.5 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA8515A (0.7 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 500 microliter of drug solution was added to 2.5 mL of a 50% PLGA8515E (0.7 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 200 microliter of drug solution was added to 2.5 mL of a 50% PLGA8515 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 50 microliter of drug solution was added to 2.5 mL of a 50% PLGA7525 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA7525 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 200 microliter of drug solution was added to 2.5 mL of a 50% PLGA7525 in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA6535A (0.3 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 100 microliter of drug solution was added to 2.5 mL of a 50% PLGA6535A (0.5 dl/g) in NMP solution using syringe to syringe mixing with a female to female luer connector. 500 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 500 microliter of drug solution was added to 2.5 mL of a 50% PLGA6535E (0.3 dl/g) in NMP solution using syringe to syringe mixing with a female to female luer connector. 250 microliters in a 1 mL syringe was loaded into a 20 G×20 cm Chiba biopsy needle with depth markers.

0.5 mL N-methyl pyrrolidone (NMP) was added to a vial with 0.25 g paclitaxel and vortexed until dissolved. 500 microliter of drug solution was added to 2.5 mL of a 50% PLGA6535E (0.5 dl/g) in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA5050A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA5050A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA5050A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA5050A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA5050A in NMP solution using syringe to syringe mixing with a female to female luer connector (#SRF1). 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA5050A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA5050E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA5050E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA5050E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA5050E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA5050E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA5050E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA8515A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA8515A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA8515A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA8515A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA8515A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA8515A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA8515E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA8515E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA8515E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA8515E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA8515E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA8515E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA6535A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA6535A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA6535A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA6535A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA5050A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA6535A in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.25 g paclitaxel and vortexed until dissolved. 400 mg of drug solution was added to 2.9 g of a 20% by weight PLGA6535E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 30% by weight PLGA6535E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA6535E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 40% by weight PLGA6535E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA6535E in NMP solution using syringe to syringe mixing with a female to female luer connector. 250 microliters in a 1 mL syringe was loaded into a 20 G×20 cm Chiba biopsy needle with depth markers.

1.08 g N-methyl pyrrolidone (NMP) was added to a vial with 0.50 g paclitaxel and vortexed until dissolved. 400 mg of drug solution was added to 2.9 g of a 50% by weight PLGA6535E in NMP solution using syringe to syringe mixing with a female to female luer connector. 250 microliters in a 1 mL syringe was loaded into a 20 G×20 cm Chiba biopsy needle with depth markers.

Additional aspects of the disclosure are set forth in the Embodiments E1-E65:

E1. A Sustained Release Formulation (SRF) for treating fibroids by dispensing no more than a unit volume of the SRF at a location in the uterus using a needle syringe, comprising: a cytostatic or cytotoxic drug; a glycolide-based bioabsorbable copolymer; and a solvent capable of dissolving the drug and copolymer.

E2. The SRF of E1 or any embodiments depending from E1, wherein the glycolide-based bioabsorbable copolymer is selected from the set of poly(D,L-lactide-co-glycolide) (50:50), poly(D,L-lactide-co-glycolide) (65:35), and poly(D,L-lactide-co-glycolide) (85:15).

E3. The SRF of E1 or any embodiments depending from E1, wherein the solvent capable of dissolving the drug and copolymer is selected from the set of N-methylpyrrolidone (NMP) and dimethyl sulfoxide (DMSO).

E4. The SRF of E1 or any embodiments depending from E1, wherein the cytotoxic drug is selected from the set of paclitaxel, docetaxel, taxanes, protaxel, vincristine, etoposide, nocodazole, indirubin, anthracycline derivatives, daunorubicin, daunomycin, plicamycin, tauromustine, bofumustane, and plicamycin, irinotecan and doxorubicin and combinations thereof.

E5. The SRF of E1 or any embodiments depending from E1, wherein the cytotoxic drug is paclitaxel.

E6. The SRF of E1 or any embodiments depending from E1, wherein the glycolide-based bioabsorbable copolymer is selected from the set of poly(D,L-lactide-co-glycolide) (PLGA) and PLGA-PEG-PLGA.

E7. The SRF of E1 or any embodiments depending from E1, wherein glycolide-based bioabsorbable copolymer has a total concentration of 25-75% by wt., the solvent has a total concentration of 75-25% by wt., and the cytotoxic or cytostatic drug has a total concentration of 0.5%-30% by wt.; 1%-20% by wt. o, or 2%-6% by wt.

E8. The SRF of E1 or any embodiments depending from E1, wherein the composition is adapted to fully release the drug into the uterus over at least 14 days from being injected into the uterus, or over a 30 to 90 day period, or over a 90 to 180 day period.

E9. The SRF of E1 or any embodiments depending from E1, wherein the drug has a release rate of no more than 10% to 85% over the first month, 25% to 95% over the first three months, and/or 50% to 100% over the first six months.

E10. The SRF of E1 or any embodiments depending from E1, wherein the drug has a release rate of between 5% to 50% during the first 24 hours from injecting the composition into the uterus.

E11. The SRF of E1 or any embodiments depending from E1, wherein the glycolide-based bioabsorbable copolymer has a viscosity of between 0.2-0.5 dL/g, or 0.2 to 0.4 dL/g or 0.2 to 0.3 dL/g and the ratio of DL-lactide to glycolide is from 50/50 up to 85/15.

E12. The SRF of E1 or any embodiments depending from E1, wherein the cytostatic or cytotoxic drug is 0.1 up to 10% by wt., 1-15% or up 20-30% by wt. of the composition.

E13. The SRF of E1 or any embodiments depending from E1, wherein the solvent comprises N-methyl-pyrrolidone, the drug is paclitaxel and the polymer is PLGA.

E14. The SRF of E1 or any embodiments depending from E1, wherein the SRF is adapted to release the drug into the uterus over at least 14 days from being injected into the uterus, or over a 30 to 90 day period, or over a 90 to 180 day period.

E15. The SRF of E1 or any embodiments depending from E1, wherein the drug is a cytostatic drug consisting of rapamycin, sirolimus, everolimus, temsirolimus, or zotarolimus.

E16. The SRF of E1 or any embodiments depending from E1, where the drug is a cytotoxic drug comprising paclitaxel or docetaxel.

E17. The SRF of E1 or any embodiments depending from E1, wherein the SRF forms into a solid material with shapes of one or more of, or any of combination of spheroids, rods, seeds or a gel when the SRF comes into contact with the uterus.

E18. An apparatus for treating a fibroid volume, FV, comprising: a composition comprising an SRF having an absolute viscosity, μ, and in an amount of at least one unit volume, v; and a syringe containing the composition in a barrel of the syringe, the syringe including a needle having a length, L, and lumen diameter, D, and adapted for being placed in fluid communication with the composition; wherein the syringe holds at least n of the unit volumes, v, n is related to FV as n=FV/(75*v) and the n unit volumes equal a total volume for treating the fibroid volume. For patients that have m fibroids that are treated in the same treatment session or at the same time (e.g., within 24 hours of each other) the total volume of SRF delivered to treat all m fibroids would be the sum of the m total volumes for the fibroids (it is expected that the n and/or unit volume v can vary among fibroids treated in a patient); and wherein the apparatus has a HIR between 2 and 1300, 10 and 400, or 30 and 300, wherein the HIR is defined as (μ)*(L)\hat{ }2/(vD)*10\hat{ }(−6).

E19. The apparatus of E18 or any embodiment depending therefrom, wherein μ is between 500 cP and 6000 cP.

E20. The apparatus of E18 or any embodiment depending therefrom, wherein L is between 15 and 30 cm.

E21. The apparatus of E18 or any embodiment depending therefrom, wherein the barrel has a slenderness ratio of between 5 and 50, or more narrowly between 10 and 30.

E22. The apparatus of E18 or any embodiment depending therefrom, wherein the needle has a slenderness ratio (L/D) of between 125 and 750, or between 400 and 750.

E23. The apparatus of E18 or any embodiment depending therefrom, wherein the needle has an inner diameter of 0.02 cm to about 0.12 cm or more narrowly 0.06 cm to 0.08 cm.

E24. The apparatus of E18 or any embodiment depending therefrom, wherein v is between 0.05 ml and 0.2 ml.

E25. The apparatus of E18 or any embodiment depending therefrom, wherein L is between 25 and 30 cm, the barrel has a slenderness ratio of about 10 and 30, the needle has an inner diameter of 0.06 cm to about 0.08 cm, v is between 0.05 ml and 0.2 ml, and the viscosity is between 1065 cP and 4000 cP.

E26. The apparatus of E18 or any embodiment depending therefrom, wherein the syringe barrel contains between 2 and 10 unit volumes of the composition, excluding dead volume of composition within the syringe barrel.

E27. The apparatus of E18 or any embodiment depending therefrom, wherein the composition comprises a bioabsorbable polymer at a total concentration of 40-50% by wt., a solvent at a total concentration of 80-20%, 75-25%, 60-40%, by wt., and a drug at a total concentration of 0.5%-30% by wt.; 1%-20% by wt. o, or 2%-6% by wt.

E28. The apparatus of E18 or any embodiment depending therefrom, wherein a drug of the SRF has a release rate of no more than 10% to 85% over the first month, 25% to 95% over the first three months, and/or 50% to 100% over the first six months.

E29. The apparatus of E18 or any embodiment depending therefrom, wherein the drug has a release rate of between 5% to 50% during the first 24 hours from injecting the composition into the uterus.

E30. The apparatus of E18 or any embodiment depending therefrom, wherein the SRF comprises a cytostatic or cytotoxic drug of 0.1 up to 10% by wt., 10-15% or up to 20-30% by wt. of the SRF.

E31. The apparatus of E18 or any embodiment depending therefrom, wherein the drug is sirolimus, docetaxel, or paclitaxel.

E32. The apparatus of E18 or any embodiment depending therefrom, wherein the drug comprises a hormone, GnRH agonist or antagonist, antibiotic, stimulant, analgesic, anesthesia, antidepressant, anti-inflammatory such as corticosteroids, contraceptive, antifibrinolytic and/or immunotherapy.

E33. The apparatus of E18 or any embodiment depending therefrom, wherein the solvent comprises N-methyl-pyrrolidone, the drug is paclitaxel and the polymer is PLGA.

E34. The apparatus of E18 or any embodiment depending therefrom, wherein the HIR is between 40 and 400.

E35. An apparatus for treating a fibroid volume, FV, comprising: a composition comprising an SRF having an absolute viscosity, μ, and in an amount of at least one unit volume, v; and a syringe containing the composition in a barrel of the syringe, the syringe including a needle having a length, L, and lumen diameter, D, and adapted for being placed in fluid communication with the composition; wherein the syringe holds at least n of the unit volumes, v, n is related to FV as n=FV/(75*v) and the n unit volumes equal a total volume for treating the fibroid volume. For patients that have m fibroids that are treated in the same treatment session or at the same time (e.g., within 24 hours of each other) the total volume of SRF delivered to treat all m fibroids would be the sum of the m total volumes for the fibroids (it is expected that the n and/or unit volume v can vary among fibroids treated in a patient); and wherein the apparatus has a HIR between 2 and 1300, 10 and 400, or 30 and 300, wherein the HIR is defined as (μ)*(L)\hat{ }2/(vD)*10\hat{ }(−6).

E36. The apparatus E18, wherein the HIR is between 30 and 300.

E37. The apparatus E18, wherein the syringe barrel contains between 2 and 10 unit volumes of the composition, excluding dead volume of composition within the syringe barrel.

E38. A method of making, comprising: combining a cytotoxic or cytostatic drug, a glycolide-based bioabsorbable copolymer, and a solvent capable of dissolving the drug and copolymer to form composition for treating fibroids;

E39. The method of making according to E48 or any embodiment depending therefrom, wherein the composition when placed within a needle syringe forms an apparatus having a HIR of between 13 and 1300.

E40. The method of making according to E48 or any embodiment depending therefrom, wherein the composition when delivered to a fibroid produces an efficacious outcome when delivered to a fibroid as a total volume.

E41. The method of making according to E48 or any embodiment depending therefrom, wherein the cytotoxic or cytostatic drug, the glycolide-based bioabsorbable copolymer, and the solvent are combined when placed within a barrel of the needle syringe.

E42. A method for treating uterine fibroids in a subject in need thereof, comprising: using a needle syringe containing a composition comprising a cytotoxic drug or cytostatic drug, a glycolide-based bioabsorbable copolymer, and a water soluble solvent capable of dissolving the drug and copolymer; and dispensing a total volume of the composition into a uterus to produce an efficacious outcome for treating fibroids, including dispensing no more than a unit volume of the composition into a first uterus location using the needle syringe.

E43. A method for administering a cytotoxic drug or cytostatic drug, comprising, using a needle syringe containing a composition comprising a cytotoxic drug or cytostatic drug, a glycolide-based bioabsorbable copolymer, and a water soluble solvent capable of dissolving the drug and copolymer; and administering an effective amount of the composition into a uterus to treat fibroids, including dispensing no more than a unit volume of the composition into a first uterus location using the needle syringe.

E44. A method for treating uterine fibroids in a subject in need thereof, comprising: using a needle syringe containing a composition comprising a cytotoxic drug or cytostatic drug, a glycolide-based bioabsorbable copolymer, and a water soluble solvent capable of dissolving the drug and copolymer; and dispensing a total volume of the composition into a uterus to produce an efficacious outcome for treating fibroids, including dispensing no more than a unit volume of the composition into a first uterus location using the needle syringe.

E45. The method of any embodiment depending from E44, wherein the apparatus has a H-Injectate Rating (HIR) between 2 and 1307, 10 and 400, or 30 and 300, wherein the HIR is defined as (μ)*(L)/(vD)*10\hat{ }(−6), wherein μ is the absolute viscosity of the composition (cP), L is the needle length (cm) as measured from the needle tip to the end of the needle hub, D is the needle lumen diameter (cm) and v is the unit volume (cm³ or cc) of the composition contained in the syringe barrel.

E46. The method of any embodiment depending from E44, further comprising injecting no more than a unit volume of the composition into a second target tissue of the uterus using the apparatus.

E47. The method of any embodiment depending from E44, wherein a plurality, n, of the no more than a unit volume, v, are injected into the uterus to treat a fibroid having a corresponding n fibroid locations, wherein n is related to a fibroid volume (FV) as n=FV/(75*v), wherein the sum of the plurality of the n of the unit volumes v is a total volume for treatment of the fibroid.

E48. The method of any embodiment depending from E44, wherein the needle syringe comprises a barrel that is configured to hold at least n of the unit volumes, such that
n is 1 when the fibroids volume is 0.05 mL to 1 mL,
n is 1 to 2 when the fibroids volume is 1 mL to 5 mL,
n is 2 to 3 when the fibroids volume is 5 mL to 10 mL, and
n is 3 to 4 when the fibroids volume is 10 mL to 15 mL.

E49. The method of any embodiment depending from E44, wherein the unit volume is 0.05 ml to 0.5 ml, 0.05 ml to 0.1 ml, 0.1 to 0.2 ml, or 0.05 to 0.2 ml.

E50. The method of any embodiment depending from E44, wherein the composition comprises between 4% by wt. to 15% by wt. of the cytotoxic or cytostatic drug.

E51. The method of E50, wherein the cytotoxic drug is docetaxel or paclitaxel.

E52. The method of E50, wherein the cytostatic drug is sirolimus.

E53. The method of any embodiment depending from E44, wherein the uterus volume or weight is measured by ultrasonic imaging, and the injection is performed using an approach selected from hysteroscopic, laparoscopic or robotic.

E54. The method of any embodiment depending from E44, wherein the composition further comprises an estrogen, progestin, levonorgestrel, GnRH agonist, GnRH antagonist, or a combination thereof.

E55. The method of any embodiment depending from E44, wherein the glycolide-based bioabsorbable copolymer is selected from the group consisting of poly(D,L-lactide-co-glycolide) (50:50), poly(D,L-lactide-co-glycolide) (65:35), and poly(D,L-lactide-co-glycolide) (75:25), and poly(D,L-lactide-co-glycolide) (85:15).

E56. The method of any embodiment depending from E44, wherein the solvent capable of dissolving the drug and copolymer is selected from the group consisting of N-methylpyrrolidone (NMP), dimethyl sulfoxide (DMSO) and combinations thereof.

E57. The method of any embodiment depending from E44, wherein the glycolide-based bioabsorbable copolymer is selected from the group consisting of poly(D,L-lactide-co-glycolide) (PLGA) and PLGA-PEG-PLGA.

E58. The method of any embodiment depending from E44, wherein glycolide-based bioabsorbable copolymer has a total concentration of 30% by wt. to 50% by wt., the solvent has a total concentration of 30% by wt. to 50% by wt., and the cytotoxic or cytostatic drug has a total concentration of 0.5% by wt. to 30% by wt.; 1% by wt. to 20% by wt., or 2% by wt. to 6% by wt.

E59. The method of any embodiment depending from E44, wherein the drug has a release rate of between 5% to 50% during the first 24 hours from injecting the composition into the uterus.

E60. The method of any embodiment depending from E44, wherein the solvent comprises N-methyl-pyrrolidone, the drug is paclitaxel and the polymer is PLGA.

E61. The method of any embodiment depending from E44, wherein the sum of the plurality of the unit volumes is the total volume for treating the fibroids.

E62. A method of treating uterine fibroids in a subject in need thereof, comprising using an apparatus comprising a needle syringe containing a composition comprising a cytotoxic or cytostatic drug, a glycolide-based bioabsorbable copolymer, and a solvent capable of dissolving the drug and copolymer, wherein the composition has an absolute viscosity, μ, and the needle syringe has a needle length L with inner diameter D; and dispensing a plurality of unit volumes, v, of the composition at a respective plurality of different locations in the uterus using the needle syringe;
wherein a unit volume is from 0.05 ml to 0.2 ml;
wherein the plurality of unit volumes is equal to a total volume for treating uterine fibroids; and
wherein the apparatus has a HIR of between 10 to 400 or 30 to 300 and HIR is defined as

$$HIR = \frac{μ {L}^{2}}{vD} ( 1 {0}^{- 6} ) .$$

E63. The method of any embodiment depending from E62, wherein the composition has between 4% by wt. to 15% by wt. of the cytotoxic or cytostatic drug, wherein the cytotoxic drug is docetaxel or paclitaxel, and wherein the cytostatic drug is sirolimus.

E64. The method of any embodiment depending from E62, wherein the plurality of unit volumes, n, is related to a fibroid volume, FV, for treatment of fibroids as n=FV/(75*v).

E65. The method of any embodiment depending from E44, further comprising measuring a size of each fibroid of the subject having a plurality of fibroids and based on the size of each of the fibroids: selecting respective total volumes, unit volumes, and locations for injecting each of the unit volumes into each of the respective fibroids, wherein the total amount of the composition injected into the subject's uterus during a single treatment for treating all fibroids in the subject is equal to the sum total of the total volumes of the composition for each of the fibroids.

1. A method for treating uterine fibroids in a subject in need thereof, comprising:
using a needle syringe containing a composition comprising a cytotoxic drug or cytostatic drug, a glycolide-based bioabsorbable copolymer, and a water soluble solvent capable of dissolving the drug and copolymer; and
dispensing a total volume of the composition into a uterus to produce an efficacious outcome for treating fibroids, including dispensing no more than a unit volume of the composition into a first uterus location using the needle syringe.

2. The method of claim 1, wherein the apparatus has a H-Injectate Rating (HIR) between 2 and 1307, 10 and 400, or 30 and 300, wherein the HIR is defined as (μ)*(L)/(vD)*10\hat{ }(−6), wherein μ is the absolute viscosity of the composition (cP), L is the needle length (cm) as measured from the needle tip to the end of the needle hub, D is the needle lumen diameter (cm) and v is the unit volume (cm³ or cc) of the composition contained in the syringe barrel.

3. The method of claim 1, further comprising injecting no more than a unit volume of the composition into a second target tissue of the uterus using the apparatus.

4. The method of claim 1, wherein a plurality, n, of the no more than a unit volume, v, are injected into the uterus to treat a fibroid having a corresponding n fibroid locations, wherein n is related to a fibroid volume (FV) as n=FV/(75*v), wherein the sum of the plurality of the n of the unit volumes v is a total volume for treatment of the fibroid.

5. The method of claim 4, wherein the needle syringe comprises a barrel that is configured to hold at least n of the unit volumes, such that
n is 1 when the fibroids volume is 0.05 mL to 1 mL,
n is 1 to 2 when the fibroids volume is 1 mL to 5 mL,
n is 2 to 3 when the fibroids volume is 5 mL to 10 mL, and
n is 3 to 4 when the fibroids volume is 10 mL to 15 mL.

6. The method of claim 1, wherein the unit volume is 0.05 ml to 0.5 ml, 0.05 ml to 0.1 ml, 0.1 to 0.2 ml, or 0.05 to 0.2 ml.

7. The method of claim 1, wherein the composition comprises between 4% by wt. to 15% by wt. of the cytotoxic or cytostatic drug.

8. The method of claim 7, wherein the cytotoxic drug is docetaxel or paclitaxel.

9. The method of claim 7, wherein the cytostatic drug is sirolimus.

10. The method of claim 9, wherein the uterus volume or weight is measured by ultrasonic imaging, and the injection is performed using an approach selected from hysteroscopic, laparoscopic or robotic.

11. The method of claim 1, wherein the composition further comprises an estrogen, progestin, levonorgestrel, GnRH agonist, GnRH antagonist, or a combination thereof.

12. The method of claim 1, wherein the glycolide-based bioabsorbable copolymer is selected from the group consisting of poly(D,L-lactide-co-glycolide) (50:50), poly(D,L-lactide-co-glycolide) (65:35), and poly(D,L-lactide-co-glycolide) (75:25), and poly(D,L-lactide-co-glycolide) (85:15).

13. The method of claim 1, wherein the solvent capable of dissolving the drug and copolymer is selected from the group consisting of N-methylpyrrolidone (NMP), dimethyl sulfoxide (DMSO) and combinations thereof.

14. The method of claim 1, wherein the glycolide-based bioabsorbable copolymer is selected from the group consisting of poly(D,L-lactide-co-glycolide) (PLGA) and PLGA-PEG-PLGA.

15. The method of claim 1, wherein glycolide-based bioabsorbable copolymer has a total concentration of 30% by wt. to 50% by wt., the solvent has a total concentration of 30% by wt. to 50% by wt., and the cytotoxic or cytostatic drug has a total concentration of 0.5% by wt. to 30% by wt.; 1% by wt. to 20% by wt., or 2% by wt. to 6% by wt.

16. The method of claim 1, wherein the drug has a release rate of between 5% to 50% during the first 24 hours from injecting the composition into the uterus.

17. The method of claim 1, wherein the solvent comprises N-methyl-pyrrolidone, the drug is paclitaxel and the polymer is PLGA.

18. The method of claim 1, wherein the sum of the plurality of the unit volumes is the total volume for treating the fibroids.

19. A method of treating uterine fibroids in a subject in need thereof, comprising
using an apparatus comprising a needle syringe containing a composition comprising a cytotoxic or cytostatic drug, a glycolide-based bioabsorbable copolymer, and a solvent capable of dissolving the drug and copolymer, wherein the composition has an absolute viscosity, μ, and the needle syringe has a needle length L with inner diameter D; and
dispensing a plurality of unit volumes, v, of the composition at a respective plurality of different locations in the uterus using the needle syringe;
wherein a unit volume is from 0.05 ml to 0.2 ml;
wherein the plurality of unit volumes is equal to a total volume for treating uterine fibroids; and
wherein the apparatus has a HIR of between 10 to 400 or 30 to 300 and HIR is defined as
$$HIR = \frac{μ {L}^{2}}{vD} ( 1 {0}^{- 6} ) .$$

20. The method of claim 19, wherein the composition has between 4% by wt. to 15% by wt. of the cytotoxic or cytostatic drug, wherein the cytotoxic drug is docetaxel or paclitaxel, and wherein the cytostatic drug is sirolimus.

21. The method of claim 19, wherein the plurality of unit volumes, n, is related to a fibroid volume, FV, for treatment of fibroids as n=FV/(75*v).

22. The method of claim 1, further comprising
measuring a size of each fibroid of the subject having a plurality of fibroids and based on the size of each of the fibroids:
selecting respective total volumes, unit volumes, and locations for injecting each of the unit volumes into each of the respective fibroids,
wherein the total amount of the composition injected into the subject's uterus during a single treatment for treating all fibroids in the subject is equal to the sum total of the total volumes of the composition for each of the fibroids.
